(19) United States
(12) Patent Application Publication
(10) Pub. No.: US 2026/0008849 A1
(43) Pub. Date: Jan. 8, 2026

(54) ANTIBODY FORMULATIONS

(71) Applicant: Oncternal Therapeutics, Inc., San Diego, CA (US)

(72) Inventors: Rajesh KRISHNAN, Carlsbad, CA (US); Weijie WANG, Fremont, CA (US)

(21) Appl. No.: 19/330,627

(22) Filed: Sep. 16, 2025

Related U.S. Application Data
(63) Continuation of application No. 18/877,628, filed on Dec. 20, 2024, now abandoned.
(60) Provisional application No. 63/367,195, filed on Jun. 28, 2022.

(51) Int. Cl. C07K 16/28; A61K 39/00; A61K 39/395; A61K 47/12; A61K 47/18; A61K 47/26

(52) U.S. Cl. CPC C07K 16/2803; A61K 39/39591; A61K 47/12; A61K 47/183; A61K 47/26; A61K 2039/545; C07K 2317/565; C07K 2317/94

(57) ABSTRACT

Provided herein are pharmaceutical compositions comprising: an anti-ROR1 antibody or ROR1-binding antibody fragment thereof; and about 5% (w/v) or greater trehalose; wherein the anti-ROR1 antibody or ROR1-binding antibody fragment thereof. The pharmaceutical compositions disclosed herein reduces the formation of aggregates, and maintains high purity of the antibody or antigen binding fragment thereof.

### CROSS-REFERENCE

This application is a continuation of U.S. patent application Ser. No. 18/877,628, filed Dec. 20, 2024, which is a U.S. National Stage Application of PCT/US2023/068932, filed Jun. 23, 2023, which claims the benefit of U.S. Provisional Application No. 63/367,195, filed Jun. 28, 2022, which each applications are incorporated herein by reference.

### INCORPORATION BY REFERENCE OF SEQUENCE LISTING

The present application is being filed along with a Sequence Listing in electronic format. The Sequence Listing is provided as a file entitled 71512-725_301_SL.xml, created Jun. 22, 2023, which is 10,871 bytes in size. The information in the electronic format of the Sequence Listing is incorporated by reference in its entirety

### BACKGROUND

Cancer is a leading cause of death worldwide. In the United States alone, cancer causes the death of well over a half-million people annually, with some 1.4 million new cases diagnosed per year. Receptor tyrosine kinases (RTKs) play critical roles in cell differentiation, proliferation, migration, angiogenesis, and survival. The receptor tyrosine kinase-like orphan receptor 1 (ROR1) is a type I membrane protein that belongs to the ROR subfamily and has extracellular domains that contain immunoglobulin (Ig)-like, Frizzled, and Kringle domains. Recent advances have shed light on the role ROR1 may play in non-canonical WNT-signaling to promote the survival of malignant cells. Studies have also shown that non-canonical WNT signaling plays a major role in basal-like and other subtypes of cancer metastasis. The ability to develop and manufacture antibodies at large scale targeting ROR1 to a level suitable for clinical application is an important step in the development of therapies targeting ROR1.

### SUMMARY

The pharmaceutical use of antibodies has increased over the past decades, where in many instances, the majority of therapeutic antibodies are either injected or infused via the intravenous (IV) route. The challenge in developing and scaling therapeutic antibodies is that, generally, the amount of antibody that can be administered via the intravenous route is limited by the physico-chemical properties of the antibody, for example, by an antibody's solubility and stability in a suitable liquid formulation and by the volume of the infusion fluid. Regardless of route, injection pathways also generally need high protein concentration in the final solution to be injected. One problem to be solved is how to develop formulations for generating highly concentrated, stable pharmaceutical formulations of a pharmaceutically active ROR1 antibodies or a mixture of such antibody molecules for injection (e.g., intravenous injection or infusion).

Provided herein are compositions useful for generating highly concentrated, stable pharmaceutical formulations of a pharmaceutically active antibody and antigen binding antibody fragments.

A composition comprising: (a) an antibody or antigen binding fragment thereof at a concentration of between about 20 and about 80 milligrams per milliliter; (b) a buffer; (c) a chelating agent; (d) a surfactant; and 5.0% (w/v) or greater of trehalose dihydrate; wherein the pH of the composition is 6.0 or lower. In certain embodiments, the antibody is an ROR1 antibody. In certain embodiments, the antibody the antibody is zilovertamab. In certain embodiments, the antibody the buffer comprises acetate, citrate, histidine, arginine, succinate, or phosphate. In certain embodiments, the antibody the buffer comprises sodium citrate. In certain embodiments, the antibody the buffer is present within the composition at a concentration from about 5 mM to about 100 mM. In certain embodiments, the antibody the buffer is present within the composition at a concentration from about 5 mM to about 50 mM. In certain embodiments, the antibody the buffer is present within the composition at a concentration from about 5 mM to about 20 mM. In certain embodiments, the antibody the buffer is present within the composition at a concentration of about 10 mM. In certain embodiments, the antibody the chelating agent comprises ethylenediaminetetraacetic acid (EDTA) or diethylenetriamine pentaacetate (DTPA). In certain embodiments, the antibody the chelating agent comprises EDTA. In certain embodiments, the antibody the chelating agent is present within the composition at a concentration from about 0.01 mM to about 0.2 mM. In certain embodiments, the antibody the chelating agent is present within the composition at a concentration from about 0.01 mM to about 0.1 mM. In certain embodiments, the antibody the chelating agent is present within the composition at a concentration from about 0.02 mM to about 0.2 mM. In certain embodiments, the antibody the chelating agent is present within the composition at a concentration of about 0.05 mM. In certain embodiments, the antibody the surfactant comprises polysorbate 80, polysorbate 20, or a polyethylene-polypropylene copolymer. In certain embodiments, the antibody the surfactant comprises polysorbate 80. In certain embodiments, the antibody the surfactant is present within the composition at a concentration from about 0.005% (w/v) to about 0.1% (w/v). In certain embodiments, the antibody the surfactant is present within the composition at a concentration from about 0.01% (w/v) to about 0.05% (w/v). In certain embodiments, the antibody the surfactant is present within the composition at a concentration from about 0.01% (w/v) to about 0.03% (w/v). In certain embodiments, the antibody the surfactant is present within the composition at a concentration of about 0.02% (w/v). In certain embodiments, the antibody the trehalose dihydrate is present within the composition at a concentration from about 5.0% (w/v) to about 10.0% (w/v). In certain embodiments, the antibody the trehalose dihydrate is present within the composition at a concentration from about 6.0% (w/v) to about 9.0% (w/v). In certain embodiments, the antibody the trehalose dihydrate is present within the composition at a concentration from about 7.0% (w/v) to about 8.0% (w/v). In certain embodiments, the antibody the trehalose dihydrate is present within the composition at a concentration of about 7.5% (w/v). In certain embodiments, the antibody the pH is from about 4.0 to about 6.0. In certain embodiments, the antibody the pH is from about 4.5 to about 6.0. In certain embodiments, the antibody the pH is from about 4.0 to about 5.5. In certain embodiments, the antibody the pH is from about 4.5 to about 5.5. In certain embodiments, the antibody the pH is from about 5.0 to about 5.5. In certain embodiments, the antibody the pH is about 5.2. In certain embodiments, the antibody the composition is a liquid. In certain embodiments, described herein is a method of treating a cancer comprising administering the composition to an individual in need thereof. In certain embodiments, the antibody the cancer is a leukemia or lymphoma. In certain embodiments, the antibody the leukemia or lymphoma is B cell leukemia or lymphoma. In certain embodiments, the antibody the leukemia or lymphoma is chronic lymphocytic leukemia (CLL). In certain embodiments, the antibody the leukemia or lymphoma is mantle cell lymphoma (MCL). In certain embodiments, the antibody the cancer is a solid tissue cancer. In certain embodiments, the antibody the solid tissue cancer is breast cancer, ovarian cancer, prostate cancer, lung cancer, pancreatic cancer, head and neck cancer, kidney cancer, colon cancer, or stomach cancer. In some embodiments, the composition reduces the formation of aggregates. In some embodiments, the composition comprises less than 2% of antibody aggregates. In some embodiments, the aggregates comprise precipitates, and the composition reduces the formation precipitates. In some embodiments, the composition comprises less than 2% of antibody aggregates after incubation at 40 C for 4 weeks. In some embodiments, the composition maintains the purity of the antibody or antigen binding fragment thereof. In some embodiments, the composition maintains at least 97% purity of the antibody or antigen binding fragment thereof. In some embodiments, the composition maintains at least 97% purity of the antibody or antigen binding fragment thereof after incubation at 40 C for 4 weeks. In some embodiments, the buffer is present within the composition at a concentration of up to 10 mM. In some embodiments, the buffer is sodium citrate.

Also described herein is a composition comprising: (a) an antibody or antigen binding fragment thereof at a concentration of about 40 milligrams per milliliter; (b) about 10 mM sodium citrate; (c) about 0.05 mM EDTA; (d) about 0.02% (w/v) polysorbate 80; and (e) about 7.5% (w/v) trehalose dihydrate; wherein the pH of the composition is about 5.2. In certain embodiments, the antibody the antibody is an ROR1 antibody. In certain embodiments, the antibody the antibody is zilovertamab.

Also described herein is a composition comprising: (i) an anti-ROR1 antibody or ROR1-binding antibody fragment thereof at a concentration of between about 20 and about 80 milligrams per milliliter (mg/mL); and (ii) about 5% (w/v) or greater trehalose; wherein the anti-ROR1 antibody or ROR1-binding antibody fragment thereof comprises: (a) a heavy chain complementarity determining region 1 (H-CDR1) comprising an amino acid sequence set forth in SEQ ID NO: 1; (b) a heavy chain complementarity determining region 2 (H-CDR2) comprising an amino acid sequence set forth in SEQ ID NO: 2; (c) a heavy chain complementarity determining region 3 (H-CDR3) comprising an amino acid sequence set forth in SEQ ID NO: 3; (d) a light chain complementarity determining region 1 (L-CDR1) comprising an amino acid sequence set forth in SEQ ID NO: 4; (e) a light chain complementarity determining region 2 (L-CDR2) comprising an amino acid sequence set forth in SEQ ID NO: 5; and/or (f) a light chain complementarity determining region 3 (L-CDR3) comprising an amino acid sequence set forth in SEQ ID NO: 6; wherein the anti-ROR1 antibody or ROR1-binding antibody fragment thereof binds ROR1. In certain embodiments, the anti-ROR1 antibody or ROR1-binding antibody fragment thereof comprises: (a) a heavy chain variable domain comprising 90% or greater sequence identity to SEQ ID NO: 7; and (b) a light chain variable domain comprising 90% or greater sequence identity to SEQ ID NO: 8. In certain embodiments, the anti-ROR1 antibody or ROR1-binding antibody fragment thereof comprises: (a) a heavy chain variable domain comprising SEQ ID NO: 7; and (b) a light chain variable domain comprising SEQ ID NO: 8. In certain embodiments, the anti-ROR1 antibody or ROR1-binding antibody fragment thereof comprises: (a) a heavy chain constant domain comprising 90% or greater sequence identity to SEQ ID NO: 11. In certain embodiments, the anti-ROR1 antibody or ROR1-binding antibody fragment thereof comprises: (a) a heavy chain domain comprising 90% or greater sequence identity to SEQ ID NO: 9; and (b) a light chain domain comprising 90% or greater sequence identity to SEQ ID NO: 10. In certain embodiments, the anti-ROR1 antibody or ROR1-binding antibody fragment thereof comprises: (a) a heavy chain domain comprising SEQ ID NO: 9; and (b) a light chain variable domain comprising SEQ ID NO: 10. In certain embodiments, the anti-ROR1 antibody is zilovertamab. In certain embodiments, the anti-ROR1 antibody or ROR1-binding antibody fragment thereof is present at a concentration of between about 30 mg/mL to about 50 mg/mL. In certain embodiments, the anti-ROR1 antibody or ROR1-binding antibody fragment thereof is present at a concentration of about 40 mg/mL. In certain embodiments, the composition comprises about 7% (w/v) or greater trehalose. In certain embodiments, the composition comprises about 7.5% (w/v) trehalose. In certain embodiments, the composition further comprises a citrate buffer. In certain embodiments, the composition comprises less than about 20 mM citrate buffer. In certain embodiments, the composition comprises less than about 15 mM citrate buffer. In certain embodiments, the composition comprises between about 5 mM to about 20 mM citrate buffer. In certain embodiments, the composition comprises about 10 mM citrate buffer. In certain embodiments, the composition is buffered to comprise a pH between about 4.5 to about 5.5. In certain embodiments, the composition further comprises a non-ionic surfactant. In certain embodiments, the non-ionic surfactant is polysorbate 80. In certain embodiments, the composition comprises about 0.010% (w/v) to about 0.050% (w/v) polysorbate 80. In certain embodiments, the composition further comprises a chelator. In certain embodiments, the chelator comprises EDTA. In certain embodiments, the composition comprises: (a) an anti-ROR1 antibody or ROR1-binding antibody fragment thereof at a concentration of about 40 mg/mL; (b) about 7.5% (w/v) trehalose dihydrate; (c) about 10 mM citrate buffer; (d) about 0.02% (w/v) polysorbate 80; and (e) about 0.05 mM EDTA. In some embodiments, the composition reduces the formation of aggregates. In some embodiments, the aggregates comprise precipitates, and the composition reduces the formation precipitates. In some embodiments, the composition comprises less than 2% of antibody aggregates. In some embodiments, the composition comprises less than 2% of antibody aggregates after incubation at 40 C for 4 weeks. In some embodiments, the composition maintains the purity of the antibody or antigen binding fragment thereof. In some embodiments, the composition maintains at least 97% purity of the antibody or antigen binding fragment thereof. In some embodiments, the composition maintains at least 97% purity of the antibody or antigen binding fragment thereof after incubation at 40 C for 4 weeks. In some embodiments, the buffer is present within the composition at a concentration of up to 10 mM. In some embodiments, the buffer is sodium citrate.

### BRIEF DESCRIPTION OF THE DRAWINGS

The novel features of the present within a composition disclosure are set forth with particularity in the appended claims. A better understanding of the features and advantages of the present within a composition disclosure will be obtained by reference to the following detailed description that sets forth illustrative embodiments, in which the principles of the invention are utilized, and the accompanying drawings of which:

FIG. 1 shows data demonstrating SEC-MALS chromatogram data of anti-ROR1 antibody formulations.

FIG. 2 shows data illustrating increased thermal stability and a reduced formation of antibody aggregates of anti-ROR1 antibody formulations.

### DETAILED DESCRIPTION

### Anti-ROR1 Antibodies

Provided herein are stable antibody formulations, and antibodies and antibody fragments that bind ROR1 in stable formulation. For example, provided herein are anti-ROR1 antibodies that, in certain instances, inhibit cancer cell growth and metastasis. In some embodiments, the anti-ROR1 antibody compositions are useful in methods for inhibiting metastasis using anti-ROR1 antibodies or antibody fragments thereof. In some embodiments, the antibody binds to the Ig-like domain, which is contiguous with the CRD domain of human ROR-1 (hROR1). In certain embodiments, the anti-ROR1 antibodies bind to an epitope mapping to amino acids 42-160 of hROR-1. In some embodiments, the anti-ROR1 antibodies bind to an epitope mapping to amino acids 130-160 of hROR-1.

Provided herein are stable pharmaceutical formulations of anti-ROR1 antibodies or ROR1-binding antibody fragments thereof comprising:
(a) a heavy chain complementarity determining region 1 (H-CDR1) comprising an amino acid sequence set forth in SEQ ID NO: 1;
(b) a heavy chain complementarity determining region 2 (H-CDR2) comprising an amino acid sequence set forth in SEQ ID NO: 2;
(c) a heavy chain complementarity determining region 3 (H-CDR3) comprising an amino acid sequence set forth in SEQ ID NO: 3;
(d) a light chain complementarity determining region 1 (L-CDR1) comprising an amino acid sequence set forth in SEQ ID NO: 4;
(e) a light chain complementarity determining region 2 (L-CDR2) comprising an amino acid sequence set forth in SEQ ID NO: 5; and/or
(f) a light chain complementarity determining region 3 (L-CDR3) comprising an amino acid sequence set forth in SEQ ID NO: 6.

Further provided are stable pharmaceutical formulations of anti-ROR1 antibodies or ROR1-binding antibody fragments thereof comprising:
(a) a heavy chain complementarity determining region 1 (H-CDR1);
(b) a heavy chain complementarity determining region 2 (H-CDR2);
(c) a heavy chain complementarity determining region 3 (H-CDR3);
(d) a light chain complementarity determining region 1 (L-CDR1);
(e) a light chain complementarity determining region 2 (L-CDR2); and/or
(f) a light chain complementarity determining region 3 (L-CDR3);
   wherein the H-CDR1, the H-CDR2, and the H-CDR3 are derived from SEQ ID NO: 7, and the L-CDR1, the L-CDR2, and the L-CDR3 are derived from SEQ ID NO: 8; and
   wherein the H-CDR1, the H-CDR2, the H-CDR3, L-CDR1, the L-CDR2, and the L-CDR3 are defined using Chothia, Kabat, IMGT, Contact, or AbM methodologies.

In some embodiments, the anti-ROR1 antibodies or ROR1-binding antibody fragments thereof comprise:
a heavy chain variable domain comprising an amino acid sequence having 70% or greater sequence identity to SEQ ID NO: 7; and
a light chain variable domain comprising an amino acid sequence having 70% or greater sequence identity to SEQ ID NO: 8.

In some embodiments, the heavy chain variable domain comprises an amino acid sequence having 80% or greater sequence identity to SEQ ID NO: 7. In some embodiments, the heavy chain variable domain comprises an amino acid sequence having 85% or greater sequence identity to SEQ ID NO: 7. In some embodiments, the heavy chain variable domain comprises an amino acid sequence having 90% or greater sequence identity to SEQ ID NO: 7. In some embodiments, the heavy chain variable domain comprises an amino acid sequence having 95% or greater sequence identity to SEQ ID NO: 7. In some embodiments, the heavy chain variable domain comprises an amino acid sequence having 98% or greater sequence identity to SEQ ID NO: 7. In some embodiments, the heavy chain variable domain comprises an amino acid sequence having 99% or greater sequence identity to SEQ ID NO: 7.

In some embodiments, the light chain variable domain comprises an amino acid sequence having 80% or greater sequence identity to SEQ ID NO: 8. In some embodiments, the light chain variable domain comprises an amino acid sequence having 85% or greater sequence identity to SEQ ID NO: 8. In some embodiments, the light chain variable domain comprises an amino acid sequence having 90% or greater sequence identity to SEQ ID NO: 8. In some embodiments, the light chain variable domain comprises an amino acid sequence having 95% or greater sequence identity to SEQ ID NO: 8. In some embodiments, the light chain variable domain comprises an amino acid sequence having 98% or greater sequence identity to SEQ ID NO: 8. In some embodiments, the light chain variable domain comprises an amino acid sequence having 99% or greater sequence identity to SEQ ID NO: 8.

In some embodiments, the anti-ROR1 antibodies or ROR1-binding antibody fragments thereof comprise:
a heavy chain variable domain comprising an amino acid sequence as set forth in SEQ ID NO: 7; and
a light chain variable domain comprising an amino acid as set forth in SEQ ID NO: 8.

In some embodiments, the anti-ROR1 antibodies or ROR1-binding antibody fragments thereof comprise a heavy chain constant domain comprising an amino acid sequence having 70% or greater sequence identity to SEQ ID NO: 11. In some embodiments, the heavy chain constant domain comprises an amino acid sequence having 80% or greater sequence identity to SEQ ID NO: 11. In some embodiments, the heavy chain constant domain comprises an amino acid sequence having 85% or greater sequence identity to SEQ ID NO: 11. In some embodiments, the heavy chain constant domain comprises an amino acid sequence having 90% or greater sequence identity to SEQ ID NO: 11. In some embodiments, the heavy chain constant domain comprises an amino acid sequence having 95% or greater sequence identity to SEQ ID NO: 11. In some embodiments, the heavy chain constant domain comprises an amino acid sequence having 98% or greater sequence identity to SEQ ID NO: 11. In some embodiments, the heavy chain constant domain comprises an amino acid sequence having 99% or greater sequence identity to SEQ ID NO: 11. In some embodiments, the anti-ROR1 antibodies or ROR1-binding antibody fragments thereof comprise a heavy chain constant domain comprising an amino acid sequence as set forth in SEQ ID NO: 11.

In some embodiments, the anti-ROR1 antibodies comprise:
a heavy chain domain comprising an amino acid sequence having 70% or greater sequence identity to SEQ ID NO: 9; and
a light chain domain comprising an amino acid sequence having 70% or greater sequence identity to SEQ ID NO: 10.

In some embodiments, the heavy chain domain comprises an amino acid sequence having 80% or greater sequence identity to SEQ ID NO: 9. In some embodiments, the heavy chain domain comprises an amino acid sequence having 85% or greater sequence identity to SEQ ID NO: 9. In some embodiments, the heavy chain domain comprises an amino acid sequence having 90% or greater sequence identity to SEQ ID NO: 9. In some embodiments, the heavy chain domain comprises an amino acid sequence having 95% or greater sequence identity to SEQ ID NO: 9. In some embodiments, the heavy chain domain comprises an amino acid sequence having 98% or greater sequence identity to SEQ ID NO: 9. In some embodiments, the heavy chain domain comprises an amino acid sequence having 99% or greater sequence identity to SEQ ID NO: 9.

In some embodiments, the light chain domain comprises an amino acid sequence having 80% or greater sequence identity to SEQ ID NO: 10. In some embodiments, the light chain domain comprises an amino acid sequence having 85% or greater sequence identity to SEQ ID NO: 10. In some embodiments, the light chain domain comprises an amino acid sequence having 90% or greater sequence identity to SEQ ID NO: 10. In some embodiments, the light chain domain comprises an amino acid sequence having 95% or greater sequence identity to SEQ ID NO: 10. In some embodiments, the light chain domain comprises an amino acid sequence having 98% or greater sequence identity to SEQ ID NO: 10. In some embodiments, the light chain domain comprises an amino acid sequence having 99% or greater sequence identity to SEQ ID NO: 10.

In some embodiments, the anti-ROR1 antibodies comprise:
a heavy chain domain comprising an amino acid sequence as set forth in SEQ ID NO: 9; and
a light chain domain comprising an amino acid as set forth in SEQ ID NO: 10. In some embodiments, the anti-ROR1 antibody is zilovertamab (previously known as cirmtuzumab).

### Pharmaceutical Formulations

Provided herein are pharmaceutical compositions comprising the anti-ROR1 antibodies described herein. A composition or pharmaceutical formulation or pharmaceutical composition generally encompasses and/or refers to a preparation which is in such form as to permit the biological activity of an active ingredient contained therein to be effective, and which contains no additional components which are unacceptably toxic to a subject to which the formulation would be administered. Pharmaceutical formulations are generally sterile (e.g., aseptic or free from all living microorganisms and their spores). The pharmaceutical compositions described and provided herein generally comprise, in some embodiments, the anti-ROR1 antibody or ROR1-binding antibody fragment compounded with one or more of: a buffer, a chelator, a surfactant, and a stabilizer (e.g., a tonicity or osmolarity adjusting agent).

### Anti-ROR1 Antibodies

The pharmaceutical compositions described and provided herein comprise the anti-ROR1 antibodies or ROR1-binding antibody fragments described herein. In some embodiments, the anti-ROR1 antibodies or ROR1-binding antibody fragments are present within a composition at a concentration between 20 and 80 milligrams (mg) per milliliter (mL). In certain embodiments, the anti-ROR1 antibodies or ROR1-binding antibody fragments are present within a composition at a concentration between 30 and 60 mg/mL. In certain embodiments, the anti-ROR1 antibodies or ROR1-binding antibody fragments are present within a composition at a concentration between 25 and 50 mg/mL. In certain embodiments, the anti-ROR1 antibodies or ROR1-binding antibody fragments are present within a composition at a concentration between 30 and 50 mg/mL In certain embodiments, the anti-ROR1 antibodies or ROR1-binding antibody fragments are present within a composition at a concentration between 35 and 45 mg/mL. In certain embodiments, the anti-ROR1 antibodies or ROR1-binding antibody fragments are present within a composition at a concentration between 40 and 50 mg/mL.

In certain embodiments, the composition comprises anti-ROR1 antibodies or ROR1-binding antibody fragments are present within a composition at a concentration of about 20 mg/mL to about 80 mg/mL. In certain embodiments, the anti-ROR1 antibodies or ROR1-binding antibody fragments are present within a composition at a concentration of about 20 mg/mL to about 25 mg/mL, about 20 mg/mL to about 30 mg/mL, about 20 mg/mL to about 35 mg/mL, about 20 mg/mL to about 40 mg/mL, about 20 mg/mL to about 45 mg/mL, about 20 mg/mL to about 50 mg/mL, about 20 mg/mL to about 55 mg/mL, about 20 mg/mL to about 60 mg/mL, about 20 mg/mL to about 65 mg/mL, about 20 mg/mL to about 70 mg/mL, about 20 mg/mL to about 80 mg/mL, about 25 mg/mL to about 30 mg/mL, about 25 mg/mL to about 35 mg/mL, about 25 mg/mL to about 40 mg/mL, about 25 mg/mL to about 45 mg/mL, about 25 mg/mL to about 50 mg/mL, about 25 mg/mL to about 55 mg/mL, about 25 mg/mL to about 60 mg/mL, about 25 mg/mL to about 65 mg/mL, about 25 mg/mL to about 70 mg/mL, about 25 mg/mL to about 80 mg/mL, about 30 mg/mL to about 35 mg/mL, about 30 mg/mL to about 40 mg/mL, about 30 mg/mL to about 45 mg/mL, about 30 mg/mL to about 50 mg/mL, about 30 mg/mL to about 55 mg/mL, about 30 mg/mL to about 60 mg/mL, about 30 mg/mL to about 65 mg/mL, about 30 mg/mL to about 70 mg/mL, about 30 mg/mL to about 80 mg/mL, about 35 mg/mL to about 40 mg/mL, about 35 mg/mL to about 45 mg/mL, about 35 mg/mL to about 50 mg/mL, about 35 mg/mL to about 55 mg/mL, about 35 mg/mL to about 60 mg/mL, about 35 mg/mL to about 65 mg/mL, about 35 mg/mL to about 70 mg/mL, about 35 mg/mL to about 80 mg/mL, about 40 mg/mL to about 45 mg/mL, about 40 mg/mL to about 50 mg/mL, about 40 mg/mL to about 55 mg/mL, about 40 mg/mL to about 60 mg/mL, about 40 mg/mL to about 65 mg/mL, about 40 mg/mL to about 70 mg/mL, about 40 mg/mL to about 80 mg/mL, about 45 mg/mL to about 50 mg/mL, about 45 mg/mL to about 55 mg/mL, about 45 mg/mL to about 60 mg/mL, about 45 mg/mL to about 65 mg/mL, about 45 mg/mL to about 70 mg/mL, about 45 mg/mL to about 80 mg/mL, about 50 mg/mL to about 55 mg/mL, about 50 mg/mL to about 60 mg/mL, about 50 mg/mL to about 65 mg/mL, about 50 mg/mL to about 70 mg/mL, about 50 mg/mL to about 80 mg/mL, about 55 mg/mL to about 60 mg/mL, about 55 mg/mL to about 65 mg/mL, about 55 mg/mL to about 70 mg/mL, about 55 mg/mL to about 80 mg/mL, about 60 mg/mL to about 65 mg/mL, about 60 mg/mL to about 70 mg/mL, about 60 mg/mL to about 80 mg/mL, about 65 mg/mL to about 70 mg/mL, about 65 mg/mL to about 80 mg/mL, or about 70 mg/mL to about 80 mg/mL. In certain embodiments, the anti-ROR1 antibodies or ROR1-binding antibody fragments are present within a composition at a concentration of about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, or about 80 mg/mL.

### Buffers

Provided herein are pharmaceutical compositions comprising the anti-ROR1 antibodies or ROR1-binding antibody fragments and a buffering agent. Buffers generally encompass and/or refer to agents which maintain the solution pH of the formulations of the invention in an acceptable range by the action of its acid/base conjugate components. Suitable pharmaceutically acceptable buffers include but are not limited to histidine buffers, citrate buffers, arginine buffers, gluconate buffers, succinate buffers, acetate buffers, lactic acid buffers, glutamate buffers, other organic acid buffers, tromethamine buffers, and phosphate buffers.

In some embodiments, the buffer comprises a citrate buffer. In certain embodiments, the citrate buffer is sodium citrate. In some embodiments, the buffer comprises an arginine buffer. In some embodiments, the buffer comprises a gluconate buffer. In some embodiments, the buffer comprises a succinate buffer. In some embodiments, the buffer comprises an acetate buffer. In some embodiments, the buffer comprises a lactic acid buffer. In some embodiments, the buffer comprises a glutamate buffer. In some embodiments, the buffer comprises a tromethamine buffer. In some embodiments, the buffer comprises a phosphate buffer.

In some embodiments, the buffer is present within a composition at a concentration of about 5 mM (0.005 M) to about 20 mM (0.020 M). In some embodiments, the buffer is present within a composition at a concentration of about 5 mM (0.005 M) to about 15 mM (0.015 M). In some embodiments, the buffer is present within a composition at a concentration of about 5 mM (0.005 M) to about 25 mM (0.025 M). In some embodiments, the buffer is present within a composition at a concentration of about 5 mM (0.005 M) to about 30 mM (0.030 M). In some embodiments, the buffer is present within a composition at a concentration of about 5 mM to about 25 mM. In some embodiments, the buffer is present within a composition at a concentration of about 5 mM to about 10 mM, about 5 mM to about 15 mM, about 5 mM to about 20 mM, about 5 mM to about 25 mM, about 10 mM to about 15 mM, about 10 mM to about 20 mM, about 10 mM to about 25 mM, about 15 mM to about 20 mM, about 15 mM to about 25 mM, or about 20 mM to about 25 mM. In some embodiments, the buffer is present within a composition at a concentration of at most about 10 mM, about 15 mM, about 20 mM, or about 25 mM.

In some embodiments, the buffer is present within a composition at a concentration of about 5 mM (0.005 M) to about 50 mM (0.050 M). In some embodiments, the buffer is present within a composition at a concentration of about 5 mM (0.005 M) to about 15 mM (0.015 M). In some embodiments, the buffer is present within a composition at a concentration of about 5 mM (0.005 M) to about 55 mM (0.055 M). In some embodiments, the buffer is present within a composition at a concentration of about 5 mM (0.005 M) to about 60 mM (0.030 M). In some embodiments, the buffer is present within a composition at a concentration of about 5 mM to about 10 mM, about 5 mM to about 15 mM, about 5 mM to about 20 mM, about 5 mM to about 50 mM, about 10 mM to about 15 mM, about 10 mM to about 20 mM, about 10 mM to about 50 mM, about 15 mM to about 20 mM, about 15 mM to about 50 mM, or about 20 mM to about 50 mM. In some embodiments, the buffer is present within a composition at a concentration of at most about 10 mM, about 15 mM, about 20 mM, or about 50 mM. In some embodiments, the buffer is present within the composition at a concentration of up to 10 mM. In some embodiments, the buffer is sodium citrate.

In some embodiments, the citrate buffer (e.g., sodium citrate) is present within a composition at a concentration of about 5 mM (0.005 M) to about 20 mM (0.020 M). In some embodiments, the citrate buffer (e.g., sodium citrate) is present within a composition at a concentration of about 5 mM (0.005 M) to about 15 mM (0.015 M). In some embodiments, the citrate buffer (e.g., sodium citrate) is present within a composition at a concentration of about 5 mM (0.005 M) to about 25 mM (0.025 M). In some embodiments, the citrate buffer (e.g., sodium citrate) is present within a composition at a concentration of about 5 mM (0.005 M) to about 30 mM (0.030 M). In some embodiments, the citrate buffer (e.g., sodium citrate) is present within a composition at a concentration of about 5 mM to about 25 mM. In some embodiments, the citrate buffer (e.g., sodium citrate) is present within a composition at a concentration of about 5 mM to about 10 mM, about 5 mM to about 15 mM, about 5 mM to about 20 mM, about 5 mM to about 25 mM, about 10 mM to about 15 mM, about 10 mM to about 20 mM, about 10 mM to about 25 mM, about 15 mM to about 20 mM, about 15 mM to about 25 mM, or about 20 mM to about 25 mM. In some embodiments, the citrate buffer (e.g., sodium citrate) is present within a composition at a concentration of at most about 10 mM, about 15 mM, about 20 mM, or about 25 mM. In some embodiments, the buffer is present within the composition at a concentration of up to 10 mM. In some embodiments, the buffer is sodium citrate.

In some embodiments, the citrate buffer (e.g., sodium citrate) is present within a composition at a concentration of about 5 mM (0.005 M) to about 50 mM (0.050 M). In some embodiments, the citrate buffer (e.g., sodium citrate) is present within a composition at a concentration of about 5 mM (0.005 M) to about 15 mM (0.015 M). In some embodiments, the citrate buffer (e.g., sodium citrate) is present within a composition at a concentration of about 5 mM (0.005 M) to about 55 mM (0.055 M). In some embodiments, the citrate buffer (e.g., sodium citrate) is present within a composition at a concentration of about 5 mM (0.005 M) to about 60 mM (0.030 M). In some embodiments, the citrate buffer (e.g., sodium citrate) is present within a composition at a concentration of about 5 mM to about 10 mM, about 5 mM to about 15 mM, about 5 mM to about 20 mM, about 5 mM to about 50 mM, about 10 mM to about 15 mM, about 10 mM to about 20 mM, about 10 mM to about 50 mM, about 15 mM to about 20 mM, about 15 mM to about 50 mM, or about 20 mM to about 50 mM. In some embodiments, the citrate buffer (e.g., sodium citrate) is present within a composition at a concentration of at most about 10 mM, about 15 mM, about 20 mM, or about 50 mM. In some embodiments, the buffer is present within the composition at a concentration of up to 10 mM. In some embodiments, the buffer is sodium citrate.

In some embodiments, a composition comprising the anti-ROR1 antibody or ROR1-binding antibody fragment comprises about 5 mM citrate buffer to about 25 mM citrate buffer. In some embodiments, a composition comprising the anti-ROR1 antibody or ROR1-binding antibody fragment comprises about 5 mM citrate buffer to about 10 mM citrate buffer, about 5 mM citrate buffer to about 20 mM citrate buffer, about 5 mM citrate buffer to about 25 mM citrate buffer, about 10 mM citrate buffer to about 20 mM citrate buffer, about 10 mM citrate buffer to about 25 mM citrate buffer, or about 20 mM citrate buffer to about 25 mM citrate buffer. In some embodiments, a composition comprising the anti-ROR1 antibody or ROR1-binding antibody fragment comprises about 5 mM citrate buffer, about 10 mM citrate buffer, about 20 mM citrate buffer, or about 25 mM citrate buffer. In some embodiments, a composition comprising the anti-ROR1 antibody or ROR1-binding antibody fragment comprises at least about 5 mM citrate buffer, about 10 mM citrate buffer, or about 20 mM citrate buffer. In some embodiments, a composition comprising the anti-ROR1 antibody or ROR1-binding antibody fragment comprises at most about 10 mM citrate buffer, about 20 mM citrate buffer, or about 25 mM citrate buffer. In some embodiments, the buffer is present within the composition at a concentration of up to 10 mM, the buffer is sodium citrate, and the reduced concentration of the citrate buffer of up to 10 mM contributes to the enhanced stability of the antibody formulation.

In some embodiments, a composition comprising the anti-ROR1 antibody or ROR1-binding antibody fragment comprises about 5 mM citrate buffer to about 60 mM citrate buffer. In some embodiments, a composition comprising the anti-ROR1 antibody or ROR1-binding antibody fragment comprises about 5 mM citrate buffer to about 10 mM citrate buffer, about 5 mM citrate buffer to about 20 mM citrate buffer, about 5 mM citrate buffer to about 30 mM citrate buffer, about 5 mM citrate buffer to about 40 mM citrate buffer, about 5 mM citrate buffer to about 50 mM citrate buffer, about 5 mM citrate buffer to about 60 mM citrate buffer, about 10 mM citrate buffer to about 20 mM citrate buffer, about 10 mM citrate buffer to about 30 mM citrate buffer, about 10 mM citrate buffer to about 40 mM citrate buffer, about 10 mM citrate buffer to about 50 mM citrate buffer, about 10 mM citrate buffer to about 60 mM citrate buffer, about 20 mM citrate buffer to about 30 mM citrate buffer, about 20 mM citrate buffer to about 40 mM citrate buffer, about 20 mM citrate buffer to about 50 mM citrate buffer, about 20 mM citrate buffer to about 60 mM citrate buffer, about 30 mM citrate buffer to about 40 mM citrate buffer, about 30 mM citrate buffer to about 50 mM citrate buffer, about 30 mM citrate buffer to about 60 mM citrate buffer, about 40 mM citrate buffer to about 50 mM citrate buffer, about 40 mM citrate buffer to about 60 mM citrate buffer, or about 50 mM citrate buffer to about 60 mM citrate buffer. In some embodiments, a composition comprising the anti-ROR1 antibody or ROR1-binding antibody fragment comprises about 5 mM citrate buffer, about 10 mM citrate buffer, about 20 mM citrate buffer, about 30 mM citrate buffer, about 40 mM citrate buffer, about 50 mM citrate buffer, or about 60 mM citrate buffer. In some embodiments, a composition comprising the anti-ROR1 antibody or ROR1-binding antibody fragment comprises at least about 5 mM citrate buffer, about 10 mM citrate buffer, about 20 mM citrate buffer, about 30 mM citrate buffer, about 40 mM citrate buffer, or about 50 mM citrate buffer. In some embodiments, a composition comprising the anti-ROR1 antibody or ROR1-binding antibody fragment comprises at most about 10 mM citrate buffer, about 20 mM citrate buffer, about 30 mM citrate buffer, about 40 mM citrate buffer, about 50 mM citrate buffer, or about 60 mM citrate buffer.

In some embodiments, a composition comprising the anti-ROR1 antibody or ROR1-binding antibody fragment comprises a pH of about 4.0 to about 6.0. In some embodiments, a composition comprising the anti-ROR1 antibody or ROR1-binding antibody fragment comprises a pH of about 4.5 to about 6.0. In some embodiments, a composition comprising the anti-ROR1 antibody or ROR1-binding antibody fragment comprises a pH of about 5.0 to about 6.0. In some embodiments, a composition comprising the anti-ROR1 antibody or ROR1-binding antibody fragment comprises a pH of about 4.5 to about 5.5. In some embodiments, a composition comprising the anti-ROR1 antibody or ROR1-binding antibody fragment comprises a pH of about 5.0 to about 5.5. In some embodiments, a composition comprising the anti-ROR1 antibody or ROR1-binding antibody fragment comprises a pH of about 5.2.

### Sugars

Provided herein are pharmaceutical compositions comprising the anti-ROR1 antibodies or ROR1-binding antibody fragments and a sugar. A sugar or saccharide generally encompasses and/or refers to molecules having the general composition (CH₂O)ₙ and derivatives thereof, including monosaccharides, disaccharides, trisaccharides, polysaccharides, sugar alcohols, reducing sugars, nonreducing sugars, etc. Examples of saccharides herein include glucose, sucrose, trehalose, lactose, fructose, maltose, dextran, glycerin, dextran, erythritol, glycerol, arabitol, sylitol, sorbitol, mannitol, mellibiose, melezitose, raffinose, mannotriose, stachyose, maltose, lactulose, maltulose, glucitol, maltitol, lactitol, iso-maltulose, etc.

Sugars can function as a stabilizer and/or tonicity/osmolarity adjusting agents. A stabilizer generally encompassed and/or refers to a pharmaceutically acceptable excipient, which protects the active pharmaceutical ingredient (e.g., antibody) and/or the formulation from chemical and/or physical degradation during manufacturing, storage, and application. Tonicity and/or osmolarity adjusting agents generally encompass and/or refer to pharmaceutically acceptable excipients that modulate the tonicity of the formulation.

In certain instances, the use of higher concentrations of a non-reducing sugar, such as trehalose (e.g., 5% or greater) increases the stability of the anti-ROR1 antibody or ROR1-binding antibody fragment. In certain instances, the use of higher concentrations of trehalose (e.g., 5% or greater) further enables the use of higher concentrations of the anti-ROR1 antibody or ROR1-binding antibody fragment. In certain other instances, the use of higher concentrations of trehalose (e.g., 5% or greater) also enables allows for the use of lower buffer (e.g., a citrate buffer) concentrations. In some embodiments, the sugar is trehalose (e.g., trehalose dihydrate). In certain embodiments, the trehalose is present within a composition at a concentration of about 5% (w/v) or greater. In certain embodiments, the trehalose is present within a composition at a concentration of about 6% (w/v) or greater. In certain embodiments, the trehalose is present within a composition at a concentration of about 7% (w/v) or greater. In certain embodiments, the trehalose is present within a composition at a concentration of about 8% (w/v) or greater. In certain embodiments, the trehalose is present within a composition at a concentration of about 9% (w/v) or greater. In certain embodiments, the trehalose is present within a composition at a concentration of about 10% (w/v) or greater. By way of example, percent (%) weight/volume (w/v) can refer to the concentration of trehalose or a form of a trehalose hydrate (e.g., trehalose·2H₂O). Such % w/v concentrations herein are calculated based upon the dihydrate, but unless stated otherwise also refer to equivalent amounts of trehalose anhydride, mono- or tri-hydrate, etc. In some embodiments, the use of higher concentrations of a non-reducing sugar, such as trehalose (e.g., 5% or greater) reduces the formation of aggregates. In some embodiments, the aggregates comprise precipitates and the use of higher concentrations of a non-reducing sugar, such as trehalose (e.g., 5% or greater) reduces the formation precipitates. In some embodiments, the use of higher concentrations of a non-reducing sugar, such as trehalose (e.g., 5% or greater) reduces the formation of aggregates after incubation at 40 C for 4 weeks. In some embodiments, the use of higher concentrations of a non-reducing sugar, such as trehalose (e.g., 5% or greater) maintains the purity of the antibody or antigen binding fragment thereof within the formulation. In some embodiments, the use of higher concentrations of a non-reducing sugar, such as trehalose (e.g., 5% or greater) maintains the purity of the antibody or antigen binding fragment thereof within the formulation under thermal stress. In some embodiments, the use of higher concentrations of a non-reducing sugar, such as trehalose (e.g., 5% or greater) maintains the purity of the antibody or antigen binding fragment thereof within the formulation at 97% purity of the antibody or antigen binding fragment thereof after incubation at 40 C for 4 weeks.

In some embodiments, a composition comprising the anti-ROR1 antibody or ROR1-binding antibody fragment comprises about 5% (w/v) trehalose to about 12% (w/v) trehalose. In some embodiments, a composition comprising the anti-ROR1 antibody or ROR1-binding antibody fragment comprises about 5% (w/v) trehalose to about 6% (w/v) trehalose, about 5% (w/v) trehalose to about 7% (w/v) trehalose, about 5% (w/v) trehalose to about 8% (w/v) trehalose, about 5% (w/v) trehalose to about 9% (w/v) trehalose, about 5% (w/v) trehalose to about 10% (w/v) trehalose, about 5% (w/v) trehalose to about 11% (w/v) trehalose, about 5% (w/v) trehalose to about 12% (w/v) trehalose, about 6% (w/v) trehalose to about 7% (w/v) trehalose, about 6% (w/v) trehalose to about 8% (w/v) trehalose, about 6% (w/v) trehalose to about 9% (w/v) trehalose, about 6% (w/v) trehalose to about 10% (w/v) trehalose, about 6% (w/v) trehalose to about 11% (w/v) trehalose, about 6% (w/v) trehalose to about 12% (w/v) trehalose, about 7% (w/v) trehalose to about 8% (w/v) trehalose, about 7% (w/v) trehalose to about 9% (w/v) trehalose, about 7% (w/v) trehalose to about 10% (w/v) trehalose, about 7% (w/v) trehalose to about 11% (w/v) trehalose, about 7% (w/v) trehalose to about 12% (w/v) trehalose, about 8% (w/v) trehalose to about 9% (w/v) trehalose, about 8% (w/v) trehalose to about 10% (w/v) trehalose, about 8% (w/v) trehalose to about 11% (w/v) trehalose, about 8% (w/v) trehalose to about 12% (w/v) trehalose, about 9% (w/v) trehalose to about 10% (w/v) trehalose, about 9% (w/v) trehalose to about 11% (w/v) trehalose, about 9% (w/v) trehalose to about 12% (w/v) trehalose, about 10% (w/v) trehalose to about 11% (w/v) trehalose, about 10% (w/v) trehalose to about 12% (w/v) trehalose, or about 11% (w/v) trehalose to about 12% (w/v) trehalose. In some embodiments, a composition comprising the anti-ROR1 antibody or ROR1-binding antibody fragment comprises about 5% (w/v) trehalose, about 6% (w/v) trehalose, about 7% (w/v) trehalose, about 8% (w/v) trehalose, about 9% (w/v) trehalose, about 10% (w/v) trehalose, about 11% (w/v) trehalose, or about 12% (w/v) trehalose. In some embodiments, a composition comprising the anti-ROR1 antibody or ROR1-binding antibody fragment comprises at least about 5% (w/v) trehalose, about 6% (w/v) trehalose, about 7% (w/v) trehalose, about 8% (w/v) trehalose, about 9% (w/v) trehalose, about 10% (w/v) trehalose, or about 11% (w/v) trehalose. Such % w/v concentrations herein are calculated based upon the dihydrate, but unless stated otherwise also refer to equivalent amounts of trehalose anhydride, mono- or tri-hydrate, etc.

Additionally included here are formulations comprising the anti-ROR1 antibody or ROR1-binding antibody fragment and a non-reducing sugar, such as trehalose, present within a composition at a concentration of at least 0.5% (w/v), at least 1% (w/v), at least 2% (w/v), at least 3% (w/v), or at least 4% (w/v). Such % w/v concentrations herein are calculated based upon the dihydrate, but unless stated otherwise also refer to equivalent amounts of trehalose anhydride, mono- or tri-hydrate, etc.

### Surfactants

Provided herein are pharmaceutical compositions comprising the anti-ROR1 antibodies or ROR1-binding antibody fragments and a surfactant (e.g., one or more surfactants). Surfactants generally encompass and/or refer to a pharmaceutically acceptable, surface-active agent. Surfactants are typically added to formulations to, in some embodiments, provide stability, reduce and/or prevent aggregation or to prevent and/or inhibit protein damage during processing conditions such as purification, filtration, freeze-drying, transportation, storage, and delivery. In some embodiments of the invention, a surfactant is useful for providing additional stability to the active ingredient (e.g., antibody). Examples of pharmaceutically acceptable surfactants include, but are not limited to, polyoxyethylen-sorbitan fatty acid esters, polyoxyethylene alkyl ethers, alkylphenylpolyoxyethylene ethers, polyoxyethylene-polyoxypropylene copolymers, and sodium dodecyl sulphate (SDS). Suitable polyoxyethylene-sorbitan fatty acid esters include polysorbate 20 (e.g., polyoxyethylene sorbitan monolaureate) and polysorbate 80 (e.g., polyoxyethylene sorbitan monooleate)

In some embodiments, the surfactant comprises a non-ionic surfactant. In some embodiments, the non-ionic surfactant is present within a composition at a concentration of about 0.005% (w/v) to about 0.1% (w/v). In some embodiments, the non-ionic surfactant is present within a composition at a concentration of about 0.005% (w/v) to about 0.1% (w/v). In some embodiments, the non-ionic surfactant is present within a composition at a concentration of about 0.005% (w/v) to about 0.01% (w/v), about 0.005% (w/v) to about 0.02% (w/v), about 0.005% (w/v) to about 0.05% (w/v), about 0.005% (w/v) to about 0.06% (w/v), about 0.005% (w/v) to about 0.08% (w/v), about 0.005% (w/v) to about 0.1% (w/v), about 0.01% (w/v) to about 0.02% (w/v), about 0.01% (w/v) to about 0.05% (w/v), about 0.01% (w/v) to about 0.06% (w/v), about 0.01% (w/v) to about 0.08% (w/v), about 0.01% (w/v) to about 0.1% (w/v), about 0.02% (w/v) to about 0.05% (w/v), about 0.02% (w/v) to about 0.06% (w/v), about 0.02% (w/v) to about 0.08% (w/v), about 0.02% (w/v) to about 0.1% (w/v), about 0.05% (w/v) to about 0.06% (w/v), about 0.05% (w/v) to about 0.08% (w/v), about 0.05% (w/v) to about 0.1% (w/v), about 0.06% (w/v) to about 0.08% (w/v), about 0.06% (w/v) to about 0.1% (w/v), or about 0.08% (w/v) to about 0.1% (w/v). In some embodiments, the non-ionic surfactant is present within a composition at a concentration of about 0.005% (w/v), about 0.01% (w/v), about 0.02% (w/v), about 0.05% (w/v), about 0.06% (w/v), about 0.08% (w/v), or about 0.1% (w/v). In some embodiments, the non-ionic surfactant is present within a composition at a concentration of about at least about 0.005% (w/v), about 0.01% (w/v), about 0.02% (w/v), about 0.05% (w/v), about 0.06% (w/v), or about 0.08% (w/v). In some embodiments, the non-ionic surfactant is present within a composition at a concentration of about at most about 0.01% (w/v), about 0.02% (w/v), about 0.05% (w/v), about 0.06% (w/v), about 0.08% (w/v), or about 0.1% (w/v).

In some embodiments, the surfactant comprises a polysorbate 80. In some embodiments, the surfactant comprises a polysorbate 80. In some embodiments, the polysorbate 80 is present within a composition at a concentration of about 0.005% (w/v) to about 0.1% (w/v). In some embodiments, the polysorbate 80 is present within a composition at a concentration of about 0.005% (w/v) to about 0.1% (w/v). In some embodiments, the polysorbate 80 is present within a composition at a concentration of about 0.005% (w/v) to about 0.01% (w/v), about 0.005% (w/v) to about 0.02% (w/v), about 0.005% (w/v) to about 0.05% (w/v), about 0.005% (w/v) to about 0.06% (w/v), about 0.005% (w/v) to about 0.08% (w/v), about 0.005% (w/v) to about 0.1% (w/v), about 0.01% (w/v) to about 0.02% (w/v), about 0.01% (w/v) to about 0.05% (w/v), about 0.01% (w/v) to about 0.06% (w/v), about 0.01% (w/v) to about 0.08% (w/v), about 0.01% (w/v) to about 0.1% (w/v), about 0.02% (w/v) to about 0.05% (w/v), about 0.02% (w/v) to about 0.06% (w/v), about 0.02% (w/v) to about 0.08% (w/v), about 0.02% (w/v) to about 0.1% (w/v), about 0.05% (w/v) to about 0.06% (w/v), about 0.05% (w/v) to about 0.08% (w/v), about 0.05% (w/v) to about 0.1% (w/v), about 0.06% (w/v) to about 0.08% (w/v), about 0.06% (w/v) to about 0.1% (w/v), or about 0.08% (w/v) to about 0.1% (w/v). In some embodiments, the polysorbate 80 is present within a composition at a concentration of about 0.005% (w/v), about 0.01% (w/v), about 0.02% (w/v), about 0.05% (w/v), about 0.06% (w/v), about 0.08% (w/v), or about 0.1% (w/v). In some embodiments, the polysorbate 80 is present within a composition at a concentration of about at least about 0.005% (w/v), about 0.01% (w/v), about 0.02% (w/v), about 0.05% (w/v), about 0.06% (w/v), or about 0.08% (w/v). In some embodiments, the polysorbate 80 is present within a composition at a concentration of about at most about 0.01% (w/v), about 0.02% (w/v), about 0.05% (w/v), about 0.06% (w/v), about 0.08% (w/v), or about 0.1% (w/v).

### Chelating Agents

Provided herein are pharmaceutical compositions comprising the anti-ROR1 antibodies or ROR1-binding antibody fragments and a chelator. A chelator generally encompasses and/or refers to a moiety or group on a molecule that binds to a metal ion through one or more donor atoms. Chelators include ethylenediaminetetraacetic acid (EDTA) and diethylenetriaminepentaacetic acid (DTPA).

In some embodiments, the chelator is present within a composition at a concentration of about 0.001 mM to about 0.350 mM. In certain embodiments, the chelator is present within a composition at a concentration of about 0.010 mM to about 0.100 mM. In certain embodiments, the chelator is present within a composition at a concentration of about 0.020 mM to about 0.080 mM. In certain embodiments, the chelator is present within a composition at a concentration of about 0.040 mM to about 0.060 mM. In certain embodiments, the chelator is present within a composition at a concentration of about 0.050 mM.

In some embodiments, the chelator is EDTA. In some embodiments, the EDTA is present within a composition at a concentration of about 0.001 mM to about 0.350 mM. In certain embodiments, the EDTA is present within a composition at a concentration of about 0.010 mM to about 0.100 mM. In certain embodiments, the EDTA is present within a composition at a concentration of about 0.020 mM to about 0.080 mM. In certain embodiments, the EDTA is present within a composition at a concentration of about 0.040 mM to about 0.060 mM. In certain embodiments, the EDTA is present within a composition at a concentration of about 0.050 mM.

In some embodiments, a composition comprising the anti-ROR1 antibody or ROR1-binding antibody fragment comprises about 0.001 mM EDTA to about 0.350 mM EDTA. In some embodiments, a composition comprising the anti-ROR1 antibody or ROR1-binding antibody fragment comprises about 0.010 mM EDTA to about 0.100 mM EDTA. In some embodiments, a composition comprising the anti-ROR1 antibody or ROR1-binding antibody fragment comprises about 0.020 mM EDTA to about 0.080 mM EDTA. In some embodiments, a composition comprising the anti-ROR1 antibody or ROR1-binding antibody fragment comprises about 0.040 mM EDTA to about 0.060 mM EDTA. In some embodiments, a composition comprising the anti-ROR1 antibody or ROR1-binding antibody fragment comprises about 0.050 EDTA

### Compositions

Provided herein are compositions (e.g., pharmaceutical compositions) comprising:
an anti-ROR1 antibody or ROR1-binding antibody fragment thereof; and
about 5% (w/v) or greater trehalose;
   wherein the anti-ROR1 antibody or ROR1-binding antibody fragment thereof comprises:
(a) a heavy chain complementarity determining region 1 (H-CDR1) comprising an amino acid sequence set forth in SEQ ID NO: 1;
(b) a heavy chain complementarity determining region 2 (H-CDR2) comprising an amino acid sequence set forth in SEQ ID NO: 2;
(c) a heavy chain complementarity determining region 3 (H-CDR3) comprising an amino acid sequence set forth in SEQ ID NO: 3;
(d) a light chain complementarity determining region 1 (L-CDR1) comprising an amino acid sequence set forth in SEQ ID NO: 4;
(e) a light chain complementarity determining region 2 (L-CDR2) comprising an amino acid sequence set forth in SEQ ID NO: 5; and/or
(f) a light chain complementarity determining region 3 (L-CDR3) comprising an amino acid sequence set forth in SEQ ID NO: 6.
   Further provided are compositions (e.g., pharmaceutical compositions) comprising:
an anti-ROR1 antibody or ROR1-binding antibody fragment thereof; and
about 5% (w/v) or greater trehalose;
   wherein the anti-ROR1 antibody or ROR1-binding antibody fragment thereof comprises:
(a) a heavy chain complementarity determining region 1 (H-CDR1);
(b) a heavy chain complementarity determining region 2 (H-CDR2);
(c) a heavy chain complementarity determining region 3 (H-CDR3);
(d) a light chain complementarity determining region 1 (L-CDR1);
(e) a light chain complementarity determining region 2 (L-CDR2); and/or
(f) a light chain complementarity determining region 3 (L-CDR3);
   wherein the H-CDR1, the H-CDR2, and the H-CDR3 are derived from SEQ ID NO: 7, and the L-CDR1, the L-CDR2, and the L-CDR3 are derived from SEQ ID NO: 8; and
   wherein the H-CDR1, the H-CDR2, the H-CDR3, L-CDR1, the L-CDR2, and the L-CDR3 are defined using Chothia, Kabat, IMGT, Contact, or AbM methodologies.
   In some embodiments, the anti-ROR1 antibody or ROR1-binding antibody fragment thereof comprises:
a heavy chain variable domain comprising an amino acid sequence having 70% or greater sequence identity to SEQ ID NO: 7; and
a light chain variable domain comprising an amino acid sequence having 70% or greater sequence identity to SEQ ID NO: 8.
   In some embodiments, the anti-ROR1 antibody comprises:
a heavy chain domain comprising an amino acid sequence having 70% or greater sequence identity to SEQ ID NO: 9; and
a light chain domain comprising an amino acid sequence having 70% or greater sequence identity to SEQ ID NO: 10.
   In some embodiments, the anti-ROR1 antibody comprises: a heavy chain constant domain comprising an amino acid sequence having 70% or greater sequence identity to SEQ ID NO: 11.

Further provided are compositions (e.g., pharmaceutical compositions) comprising:
an anti-ROR1 antibody or ROR1-binding antibody fragment thereof; and
about 5% (w/v) or greater trehalose;
   wherein the anti-ROR1 antibody or ROR1-binding antibody fragment thereof comprises:
a heavy chain variable domain comprising an amino acid sequence as set forth in SEQ ID NO: 7; and
a light chain variable domain comprising an amino acid sequence as set forth in SEQ ID NO: 8.

Also provided herein are compositions (e.g., pharmaceutical compositions) comprising:
an anti-ROR1 antibody; and
about 5% (w/v) or greater trehalose;
   wherein the anti-ROR1 antibody comprises:
a heavy chain domain comprising an amino acid sequence as set forth in SEQ ID NO: 9; and
a light chain domain comprising an amino acid sequence as set forth in SEQ ID NO: 10.
   Further provided are compositions (e.g., pharmaceutical compositions) comprising zilovertamab and 5% (w/v) or greater trehalose;

In some embodiments, the composition comprises the anti-ROR1 antibody or ROR1-binding antibody fragment thereof at a concentration of between about 20 mg/mL to about 80 mg/mL. In some embodiments, the composition comprises the anti-ROR1 antibody or ROR1-binding antibody fragment thereof at a concentration of between about 20 mg/mL to about 60 mg/mL. In some embodiments, the composition comprises the anti-ROR1 antibody or ROR1-binding antibody fragment thereof at a concentration of between about 30 mg/mL to about 50 mg/mL. In some embodiments, the composition comprises the anti-ROR1 antibody or ROR1-binding antibody fragment thereof at a concentration of between about 40 mg/mL to about 50 mg/mL. In some embodiments, the composition comprises the anti-ROR1 antibody or ROR1-binding antibody fragment thereof at a concentration of between about 20 mg/mL to about 80 mg/mL. In some embodiments, the composition comprises the anti-ROR1 antibody or ROR1-binding antibody fragment thereof at a concentration of about 40 mg/mL.

In some embodiments, the composition comprises about 6% (w/v) or greater trehalose. In some embodiments, the composition comprises about 7% (w/v) or greater trehalose. In some embodiments, the composition comprises about 8% (w/v) or greater trehalose. In some embodiments, the composition comprises about 9% (w/v) or greater trehalose. In some embodiments, the use of higher concentrations of a non-reducing sugar, such as trehalose (e.g., 5, 6, 7, 8, 9% w/v or greater) reduces the formation of aggregates. In some embodiments, the aggregates comprise precipitates and the use of higher concentrations of a non-reducing sugar, such as trehalose (e.g., 5, 6, 7, 8, 9% w/v or greater) reduces the formation precipitates. In some embodiments, the use of higher concentrations of a non-reducing sugar, such as trehalose (e.g., 5, 6, 7, 8, 9% w/v or greater) reduces the formation of aggregates after incubation at 40 C for 4 weeks. In some embodiments, the use of higher concentrations of a non-reducing sugar, such as trehalose (e.g., 5, 6, 7, 8, 9% w/v or greater) maintains the purity of the antibody or antigen binding fragment thereof within the formulation. In some embodiments, the use of higher concentrations of a non-reducing sugar, such as trehalose (e.g., 5, 6, 7, 8, 9% w/v or greater) maintains the purity of the antibody or antigen binding fragment thereof within the formulation under thermal stress. In some embodiments, the use of higher concentrations of a non-reducing sugar, such as trehalose (e.g., 5, 6, 7, 8, 9% w/v or greater) maintains the purity of the antibody or antigen binding fragment thereof within the formulation at 97% purity of the antibody or antigen binding fragment thereof after incubation at 40 C for 4 weeks.

In some embodiments, the composition further comprises a buffer (e.g., 5-20 mM). In certain embodiments, the buffer is a citrate buffer (e.g., sodium citrate). In certain embodiments, the composition comprises between about 5 mM to about 20 mM citrate buffer. In certain embodiments, the composition comprises less than 20 mM citrate buffer. In certain embodiments, the composition comprises less than 15 mM citrate buffer. In certain embodiments, the composition comprises about 10 mM citrate buffer.

In some embodiments, the composition is buffered to comprise a pH between about 4.0 to about 6.0. In certain embodiments, the composition is buffered to comprise a pH between about 4.5 to about 6.0. In certain embodiments, the composition is buffered to comprise a pH between about 5.0 to about 6.0. In certain embodiments, the composition is buffered to comprise a pH between about 4.5 to about 5.5. In certain embodiments, the composition is buffered to comprise a pH between about 5.0 to about 5.5. In certain embodiments, the composition is buffered to comprise a pH of about 5.2.

In some embodiments, the composition further comprises a surfactant (e.g., 0.005-0.100% (w/v)). In certain embodiments, the surfactant comprises a non-ionic surfactant (e.g., 0.005-0.100% (w/v)) . . . . In certain embodiments, the surfactant comprises polysorbate 80. In certain embodiments the composition comprises about 0.005% (w/v) to about 0.100% (w/v) polysorbate 80. In certain embodiments the composition comprises about 0.010% (w/v) to about 0.080% (w/v) polysorbate 80. In certain embodiments the composition comprises about 0.010% (w/v) to about 0.050% (w/v) polysorbate 80. In certain embodiments the composition comprises about 0.01% (w/v) to about 0.030% (w/v) polysorbate 80. In certain embodiments the composition comprises about 0.015% (w/v) to about 0.025% (w/v) polysorbate 80. In certain embodiments the composition comprises about 0.020% (w/v) polysorbate 80.

In some embodiments, the composition further comprises a chelator (e.g., 0.01-0.10 mM). In some embodiments, the chelator comprises EDTA. In certain embodiments, the composition comprises about 0.001 mM to about 0.350 mM EDTA. In certain embodiments, the composition comprises about 0.010 mM to about 0.100 mM EDTA. In certain embodiments, the composition comprises about 0.020 mM to about 0.080 mM EDTA. In certain embodiments, the composition comprises about 0.040 mM to about 0.060 mM EDTA. In certain embodiments, the composition comprises about 0.050 mM EDTA.

In some embodiments, the composition comprises:
the anti-ROR1 or ROR1-binding antibody fragment thereof at a concentration of about 40 milligrams per milliliter;
about 10 mM sodium citrate;
about 0.05 mM EDTA;
about 0.02% (w/v) polysorbate 80; and
about 7.5% (w/v) trehalose (e.g., trehalose·2H₂O);
wherein the pH of the composition is about 5.2.

In some embodiments, the composition is a for use in an intravenous injection. In some embodiments, the composition is administered intravenously. Other injection routes can be contemplated, for example, subcutaneous, intraperitoneal, intramuscular, intratumoral, or intracerebral, etc.

### Methods

The compositions described herein can be useful and/or advantageous in methods of treating cancer. Provided herein are methods of treating cancer or a tumor, the method comprising:
administering the compositions described herein comprising the ROR1 antibody or ROR1-binding fragment thereof to an individual in need thereof.
   For example, provided methods of treating cancer or a tumor, the method comprising:
administering a composition comprising an anti-ROR1 antibody or ROR1-binding antibody fragment thereof; and about 5% (w/v) or greater trehalose;
   wherein the anti-ROR1 antibody or ROR1-binding antibody fragment thereof comprises:
(a) a heavy chain complementarity determining region 1 (H-CDR1) comprising an amino acid sequence set forth in SEQ ID NO: 1;
(b) a heavy chain complementarity determining region 2 (H-CDR2) comprising an amino acid sequence set forth in SEQ ID NO: 2;
(c) a heavy chain complementarity determining region 3 (H-CDR3) comprising an amino acid sequence set forth in SEQ ID NO: 3;
(d) a light chain complementarity determining region 1 (L-CDR1) comprising an amino acid sequence set forth in SEQ ID NO: 4;
(e) a light chain complementarity determining region 2 (L-CDR2) comprising an amino acid sequence set forth in SEQ ID NO: 5; and/or
(f) a light chain complementarity determining region 3 (L-CDR3) comprising an amino acid sequence set forth in SEQ ID NO: 6.

In some embodiments, the cancer is a leukemia or a lymphoma. In certain embodiments, the leukemia or lymphoma is a B cell leukemia or lymphoma. In certain embodiments, the leukemia or lymphoma is chronic lymphocytic leukemia (CLL). In certain embodiments, the leukemia or lymphoma is mantle cell lymphoma (MCL)

In some embodiments, the cancer is a solid tissue cancer and/or comprises a solid tumor. In certain embodiments, the solid tissue cancer is breast cancer, ovarian cancer, prostate cancer, lung cancer, pancreatic cancer, head and neck cancer, kidney cancer, colon cancer, or stomach cancer.

### Definitions

An antibody is used in the broadest sense, and generally encompasses and/or refers to various antibody structures, including but not limited to monoclonal antibodies, polyclonal antibodies, multispecific antibodies (e.g., bispecific antibodies), and antibody fragments so long as they exhibit the desired antigen-binding activity. In some embodiments, an antibody or antibodies include intact antibodies and functional (antigen-binding) antibody fragments thereof, including fragment antigen binding (Fab) fragments, F(ab′)₂ fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. In some embodiments, an antibody or antibodies include genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. In some embodiments, an antibody or antibodies encompass functional antibody fragments thereof. In some embodiments, an antibody or antibodies encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region. In some embodiments, an antibody or antibodies include, but is not limited to, full-length and native antibodies, as well as fragments and portion thereof retaining the binding specificities thereof, such as any specific binding portion thereof including those having any number of, immunoglobulin classes and/or isotypes (e.g., IgG1, IgG2, IgG3, IgG4, IgM, IgA, IgD, IgE and IgM); and biologically relevant (antigen-binding) fragments or specific binding portions thereof, including but not limited to Fab, F(ab′)₂, Fv, and scFv (single chain or related entity). A monoclonal antibody is generally one within a composition of substantially homogeneous antibodies; thus, any individual antibodies comprised within the monoclonal antibody composition are identical except for possible naturally occurring mutations that can be present within a composition in minor amounts. A monoclonal antibody can comprise a human IgG1 constant region or a human IgG4 constant region.

A native antibody generally encompasses and/or refers to naturally occurring immunoglobulin molecules with varying structures. For example, native IgG antibodies can be monomeric or multimeric glycoproteins, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody can be assigned to one of two types, called kappa (κ) and lambda (λ), based on the amino acid sequence of its constant domain.

A full-length antibody, intact antibody, and whole antibody are interchangeable, and generally include and/or refer to an antibody having a structure substantially similar to a native antibody structure or having heavy chains that contain an Fc region as defined herein.

A human consensus framework generally encompasses and/or refers to a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In some embodiments, for the VL, the subgroup is subgroup kappa I as in Kabat et al., supra. In some embodiments, for the VH, the subgroup is subgroup III as in Kabat et al., supra. A humanized antibody encompasses and refers to a chimeric antibody comprising amino acid residues from non-human HVRs and amino acid residues from human framework regions (FRs). In some embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally encompasses at least a portion of an antibody constant region derived from a human antibody. A “humanized form” of an antibody, e.g., a non-human antibody, refers to an antibody that has undergone humanization.

A complementarity determining region or CDR, which are synonymous with hypervariable region or HVR, generally include and/or refer to regions of an antibody which are hypervariable in sequence and/or form structurally defined loops (hypervariable loops) and/or contain the antigen-contacting residues (antigen contacts). In some embodiments, complementarity determining regions or CDRs generally include and refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3).

A framework region or FR generally includes and/or refers to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). In some embodiments, the framework regions are defined by the non-CDR sequences of a variable region sequence. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27 (1): 55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309 (3): 657-70, (“Aho” numbering scheme); and Whitelegg N R and Rees A R, “WAM: an improved algorithm for modelling antibodies on the WEB,” Protein Eng. 2000 December; 13 (12): 819-24 (“AbM” numbering scheme. In certain embodiments, the CDRs of the antibodies described herein can be defined by a method selected from Kabat, Chothia, IMGT, Aho, AbM, or combinations thereof. In some embodiments, the CDRs and FRs are defined by and/or according to a Kabat numbering scheme. In some embodiments, the CDRs and FRs are defined by and/or according to a Chothia numbering scheme. In some embodiments, the CDRs and FRs are defined by and/or according to a IMGT numbering scheme. In some embodiments, the CDRs and FRs are defined by and/or according to an EU numbering scheme.

The boundaries of a given CDR or FR, in certain instances, vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

Fc region generally encompasses and/or refers to a C-terminal region of an immunoglobulin heavy chain that contains at least a portion of the constant region. The term includes native sequence Fc regions and variant Fc regions. In some embodiments, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

A human antibody generally encompasses and/or refers to an antibody which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

An acceptor human framework region generally encompasses and/or refers to a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework can comprise the same amino acid sequence thereof, or it can contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

A variable region or variable domain generally encompasses and/or refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs (See e.g., Kindt et al. Kuby Immunology, 6th ed., W.H. Freeman and Co., page 91 (2007)). A single V_{H} or V_{L} domain can be sufficient to confer antigen-binding specificity. Furthermore, antibodies that bind a particular antigen can be isolated using a V_{H} or V_{L} domain from an antibody that binds the antigen to screen a library of complementary V_{L} or V_{H} domains, respectively (See e.g., Portolano et al., J. Immunol. 150:880-887 (1993); Clarkson et al., Nature 352:624-628 (1991)).

Affinity generally encompasses and/or refers to the strength of the sum total of noncovalent interactions between a single binding site of a molecule (e.g., an antibody) and its binding partner (e.g., an antigen). Unless indicated otherwise, as used herein, binding affinity generally encompasses and refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary embodiments for measuring binding affinity are described throughout.

An affinity matured antibody generally encompasses and/or refers to an antibody with one or more alterations in one or more hypervariable regions (HVRs), compared to a parent antibody which does not possess such alterations, such alterations resulting in an improvement in the affinity of the antibody for antigen.

Binding and a determination of binding can be readily determined by methods known within the art (e.g., ELISA, surface plasmon resonance, bio-layer interferometry, isothermal calorimetry, etc.). In some embodiments, binding is determined by ELISA. In some embodiments, binding comprising a K_{D} less than, e.g., 10\hat{ }-5 M (10 uM) as measured by surface plasmon resonance, bio-layer interferometry, or isothermal calorimetry. In some embodiments, binding comprising a K_{D} less than, e.g., 10\hat{ }-6 M (1 uM) surface plasmon resonance, bio-layer interferometry, or isothermal calorimetry. In some embodiments, binding comprising a K_{D} less than, e.g., 10\hat{ }-7 M (100 nM) surface plasmon resonance, bio-layer interferometry, or isothermal calorimetry.

A chimeric antibody generally encompasses and/or refers to an antibody in which a portion of the heavy and/or light chain is derived from a particular source or species, while the remainder of the heavy and/or light chain is derived from a different source or species. 1 The “class” of an antibody refers to the type of constant domain or constant region possessed by its heavy chain. There are five major classes of antibodies: IgA, IgD, IgE, IgG, and IgM, and several of these can be further divided into subclasses (isotypes), e.g., IgG₁, IgG₂, IgG₃, IgG₄, IgA₁, and IgA₂. The heavy chain constant domains that correspond to the different classes of immunoglobulins are called a, d, e, g, and m, respectively.

A monoclonal antibody generally encompasses and/or refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present within a composition in minor amounts. In contrast to polyclonal antibody preparations, which typically include different antibodies directed against different determinants (epitopes), each monoclonal antibody of a monoclonal antibody preparation is directed against a single determinant on an antigen. In some embodiments, the modifier monoclonal indicates the character of the antibody as being obtained from a substantially homogeneous population of antibodies, and is not to be construed as requiring production of the antibody by any particular method. For example, the monoclonal antibodies to be used in accordance with the present within a composition disclosure can be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

An isolated antibody generally encompasses and/or refers to an antibody that has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC).

In certain embodiments, the antibody comprises one or more naturally occurring amino acids. In certain embodiments, the antibody consists of naturally occurring amino acids. As used herein, naturally occurring amino acids include and/or refer to amino acids which are generally found in nature and are not manipulated by man. In certain instances, naturally occurring includes and/or further refers to the 20 conventional amino acids: alanine (A or Ala), cysteine (C or Cys), aspartic acid (D or Asp), glutamic acid (E or Glu), phenylalanine (F or Phe), glycine (G or Gly), histidine (H or His), isoleucine (I or Ile), lysine (K or Lys), leucine (L or Leu), methionine (M or Met), asparagine (N or Asn), proline (P or Pro), glutamine (Q or Gln), arginine (R or Arg), serine (S or Ser), threonine (T or Thr), valine (V or Val), tryptophan (W or Trp), and tyrosine (Y or Tyr).

In some embodiments, the antibody comprises a variant sequence of the antibody. In certain instances, amino acid substitutions can be made in the sequence of any of the antibodies described herein, without necessarily decreasing or ablating its activity (as measured by, e.g., the binding or functional assays described herein). Accordingly, in some embodiments, the variant sequence comprises one or more amino acid substitutions. In some embodiments, the variant sequence comprises one or more substitutions in one or more CDRs. In certain embodiments, the variant sequence comprises one amino acid substitution. In certain embodiments, the variant sequence comprises two amino acid substitutions. In certain embodiments, the variant sequence comprises three amino acid substitutions. In certain instances, substitutions include conservative substitutions (e.g., substitutions with amino acids of comparable chemical characteristics). In certain instances, a non-polar amino acid can be substituted and replaced with another non-polar amino acid, wherein non-polar amino acids include alanine, leucine, isoleucine, valine, glycine, proline, phenylalanine, tryptophan and methionine. In certain instances, a neutrally charged polar amino acids can be substituted and replaced with another neutrally charged polar amino acid, wherein neutrally charged polar amino acids include serine, threonine, cysteine, tyrosine, asparagine, and glutamine. In certain instances, a positively charged amino acid can be substituted and replaced with another positively charged amino acid, wherein positively charged amino acids include arginine, lysine and histidine. In certain instances, a negatively charged amino acid can be substituted and replaced with another negatively charged amino acid, wherein negatively charged amino acids include aspartic acid and glutamic acid. Examples of amino acid substitutions also include substituting an L-amino acid for its corresponding D-amino acid, substituting cysteine for homocysteine or other non-natural amino acids.

In certain embodiments, the antibody comprises one or more non-natural amino acids. In certain embodiments, the antibody consists of non-natural amino acids. As used herein, non-natural amino acids and/or unnatural amino acids include and/or refer to amino acid structures that cannot be generated biosynthetically in any organism using unmodified or modified genes from any organism. For example, these include, but are not limited to, modified amino acids and/or amino acid analogues that are not one of the 20 naturally occurring amino acids (e.g., non-natural side chain variant sequence amino acids), D-amino acids, homo amino acids, beta-homo amino acids, N-methyl amino acids, alpha-methyl amino acids, or. By way of further example, non-natural amino acids also include 4-Benzoylphenylalanine (Bpa), Aminobenzoic Acid (Abz), Aminobutyric Acid (Abu), Aminohexanoic Acid (Ahx), Aminoisobutyric Acid (Aib), Citrulline (Cit), Diaminobutyric Acid (Dab), Diaminopropanoic Acid (Dap), Diaminopropionic Acid (Dap), Gamma-Carboxyglutamic Acid (Gla), Homoalanine (Hala), Homoarginine (Harg), Homoasparagine (Hasn), Homoaspartic Acid (Hasp), Homocysteine (Hcys), Homoglutamic Acid (Hglu), Homoglutamine (Hgln), Homoisoleucine (Hile), Homoleucine (Hleu), Homomethionine (Hmet), Homophenylalanine (Hphe), Homoserine (Hser), Homotyrosine (Htyr), Homovaline (Hval), Hydroxyproline (Hyp), Isonipecotic Acid (Inp), N aphthylalanine (Nal), Nipecotic Acid (Nip), Norleucine (Nle), Norvaline (Nva), Octahydroindole-2-carboxylic Acid (Oic), Penicillamine (Pen), Phenylglycine (Phg), Pyroglutamic Acid (Pyr), Sarcosine (Sar), tButylglycine (Tle), and Tetrahydro-isoquinoline-3-carboxylic Acid (Tic). Such non-natural amino acid residues can be introduced by substitution of naturally occurring amino acids, and/or by insertion of non-natural amino acids into the naturally occurring antibody sequence. A non-natural amino acid residue also can be incorporated such that a desired functionality is imparted to the apelin molecule, for example, the ability to link a functional moiety (e.g., PEG).

ROR1 generally refers to and includes a human ROR1 protein or gene encoding a ROR1 protein (synonyms: tyrosine-protein kinase transmembrane receptor ROR1, EC=2.7.10.1, neurotrophic tyrosine kinase, receptor-related 1, UniPrtKB Q01973), which is a tyrosine-protein kinase receptor. The extracellular domain of ROR1 consists according to of amino acids 30-406. An antibody against ROR1 or anti-ROR1 antibody generally refers to and includes an antibody that specifically binds to human ROR1 (e.g., ELISA, surface plasmon resonance, bio-layer interferometry, isothermal calorimetry, etc.). In certain embodiments, the antibody binds specifically to the extracellular domain of ROR1. In certain embodiments, the antibody binds specifically to fragments of the extracellular domain, which are the Ig-like C2-type domain, the frizzled domain, or the kringle domain. These fragments are mentioned in WO2005100605. It is further disclosed that the antibody may bind specifically to the extracellular domain fragment WNISSELNKDSYLTL of ROR1. This fragment is disclosed in Daneshmanesh A H et al., Int. J. Cancer, 123 (2008) 1190-1195.

A stable formulation refers to and/or encompasses a formulation wherein the protein (e.g., antibody) therein essentially retains its physical stability and/or chemical stability and/or biological activity upon storage at an intended storage temperature, e.g., 2-8° C. In some embodiments, the formulation essentially retains its physical and chemical stability, as well as its biological activity upon storage. A storage period can generally be selected based on the intended shelf-life of the formulation. Furthermore, the formulation is generally stable following freezing (to, e.g., −20° C.) and thawing of the formulation, for example following 1 or more cycles of freezing and thawing. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993), for example. Stability can be measured at a selected temperature for a selected time period. Stability can be evaluated qualitatively and/or quantitatively in a variety of different ways, including evaluation of aggregate formation (for example using size exclusion chromatography, by measuring turbidity, and/or by visual inspection); by assessing charge heterogeneity using cation exchange chromatography or capillary zone electrophoresis; SDS-PAGE analysis to compare reduced and intact antibody; evaluating biological activity or antigen binding function of the antibody; and the methods described herein. Instability can involve any one or more of: aggregation, deamidation (e.g. Asn deamidation), oxidation (e.g. Met oxidation), isomerization (e.g. Asp isomerisation), clipping/hydrolysis/fragmentation (e.g. hinge region fragmentation), succinimide formation, unpaired cysteine(s), etc.

A pharmaceutically acceptable carrier generally encompasses and/or refers to an ingredient in a pharmaceutical formulation, other than an active ingredient, which is nontoxic to a subject. A pharmaceutically acceptable carrier encompasses, but is not limited to, a buffer, excipient, stabilizer, or preservative.

A polypeptide or protein are used interchangeably, and generally encompass and/or refer to a polymer of amino acid residues, and are not limited to a minimum length. Polypeptides, including the provided antibodies and antibody chains and other peptides, e.g., linkers and binding peptides, can include amino acid residues including natural and/or non-natural amino acid residues. The terms also include post-expression modifications of the polypeptide, for example, glycosylation, sialylation, acetylation, phosphorylation, and the like. In some embodiments, the polypeptides can contain modifications with respect to a native or natural sequence, as long as the protein maintains the desired activity. These modifications can be deliberate, as through site-directed mutagenesis, or can be accidental, such as through mutations of hosts which produce the proteins or errors due to PCR amplification.

ROR1 expressing cancer or ROR1 expressing cancer cell generally refers to and includes to all neoplastic cell growth and proliferation, whether malignant or benign, including all transformed cells and tissues and all cancerous cells and tissues, that express, over-express, or abnormally express ROR1.

The determination of percent identity or percent similarity between two sequences can be accomplished using a mathematical algorithm. A non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul et al., 1990, J. Mol. Biol. 215:403-410. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. Additional algorithms for sequence analysis are known in the art and include ADVANCE and ADAM as described in Torellis and Robotti, 1994, Comput. Appl. Biosci. 10:3-5; and FASTA described in Pearson and Lipman, 1988, Proc. Natl. Acad. Sci. USA 85:2444-8. Alternatively, sequence alignment may be carried out using the CLUSTAL algorithm (e.g., as provided in the program Clustal-omega), as described by Higgins et al., 1996, Methods Enzymol. 266:383-402.

As used herein the term individual, patient, or subject generally includes and/or refers to individuals diagnosed with, suspected of being afflicted with, or at-risk of developing at least one disease, condition, or status for which the described compositions and method are useful for treating. In certain embodiments, the individual is a mammal. In certain embodiments, the mammal is a mouse, rat, rabbit, dog, cat, horse, cow, sheep, pig, goat, llama, alpaca, or yak. In certain embodiments, the individual is a human.

As used herein, treatment or treating generally include and/or refer to a pharmaceutical or other intervention regimen used for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made. Skilled artisans will recognize that given a population of potential individuals for treatment not all will respond or respond equally to the treatment. Such individuals are considered treated.

As used herein, the words modulation and regulation can be used interchangeably herein. In some embodiments, modulation comprises a decrease and/or reduction and/or inhibition. In some embodiments, modulation comprises to an increase and/or induction and/or promotion and/or activation.

As used herein, the words comprising (and any form of comprising, such as comprise and comprises), having (and any form of having, such as have and has), including (and any form of including, such as include and includes) or containing (and any form of containing, such as contain and contains), are inclusive or open-ended and do not exclude additional, unrecited elements or process steps. As also used herein, in any instance or embodiment described herein, comprising may be replaced with consisting essentially of and/or consisting of. used herein, in any instance or embodiment described herein, comprises may be replaced with consists essentially of and/or consists of.

As used herein, the term about in the context of a given value or range includes and/or refers to a value or range that is within 20%, within 10%, and/or within 5% of the given value or range.

As used herein, the term and/or is to be taken as specific disclosure of each of the two specified features or components with or without the other. For example, A and/or B is to be taken as specific disclosure of each of (i) A, (ii) B and (iii) A and B, just as if each were set out individually herein.

### EXAMPLES

### Example 1—Formulation

Antibody drug substance (ADS) from a GMP 2000 liter (L) was produced from a new Master Cell Bank (MCB). ADS manufacturing process and formulation are indicated in TABLE 1, which includes new Formulation 1.

TABLE 1

| Formu- | | Protein | |
|---|---|---|---|
| lation | ADS | Conc. | Buffer Composition |
| 1 | Anti-ROR1 | 40 mg/mL | 10 mM Na-citrate |
| (new) | Antibody | (Tested) | 0.05 mM EDTA |
| | H-CDR1-3 | | 7.5% (w/v) Trehalose•2H₂O |
| | SEQ ID NOs: 1-3 | | 0.02% (w/v) Polysorbate 80 |
| | L-CDR1-3 | | pH 5.2 |
| 2 | SEQ ID NOs. 4-6 | Targeted | 50 mM Na-Citrate |
| | See also: | 40 mg/mL | 0.05 mM EDTA |
| | SEQ ID NOs: | | 1% (w/v) Trehalose•2H₂O |
| | 9 and 10 | | 0.02% (w/v) Polysorbate 80 |
| | | | pH 5.2 |

A comparability study of these two samples was performed, with emphasis on the following attributes: primary structure, post-translational modifications, secondary/tertiary structure, disulfide bond, free sulfhydryl content, binding affinity, charge heterogeneity by iCIEF, and size heterogeneity by CE-SDS and SEC-MALS. Furthermore, a thermal stress study at 40° C. was performed to support the comparability assessment of these two batches of DS. Degradation trends were compared with the analysis using SE-HPLC, iCIEF, non-reduced CE-SDS, reduced CE-SDS, and binding ELISA.

### Example 2—Intact Mass Analysis

Intact mass analysis provides important information on sequence integrity and major post-translational modifications. For the molecular weight analysis of intact proteins by LC-MS, samples were diluted to 0.4 mg/mL with water and followed by UPLC separation and Xevo G2-XS Q-TOF MS detection. For the deconvoluted intact mass of both formulation samples, the major species observed corresponds to the intact ADS with the modification of the Gln to pyro-Glu (Q to pE), glycosylation (GOF, GIF), and the loss of C-terminal Lys on each heavy chain.

Masses of other expected glycoforms were also confirmed. All identified and assigned species showed measured masses within 100.0 ppm of the theoretical masses, indicating that the intact masses were consistent with the theoretical masses. ADS formulation 2 peaks of half antibody (LC/HC) were observed (TABLE 2-Sample ID 2, italicized) but there were no half antibody (LC/HC) peaks present in the ADS formulation 1 sample (TABLE 2-Sample ID 1) having increased (7.5-fold) trehalose.

TABLE 2

| | | Theor. | Measured | | Mass |
|---|---|---|---|---|---|
| | | Mass | Mass | Difference | Error |
| ID | Modification | (Da) | (Da) | (Da) | (ppm) |
| 1 | Q->pE/Q->pE, K Loss/K Loss, | 147885.5 | 147887.4 | 1.9 | 13.1 |
| | G0F/G1F | | | | |
| | Q->pE/Q->pE, K Loss/K Loss, | 148047.6 | 148047.4 | −0.3 | −1.9 |
| | G1F/G1F OR G0F/G2F | | | | |
| | Q->pE/Q->pE, K Loss/K Loss, | 147723.4 | 147728.5 | 5.1 | 34.6 |
| | G0F/G0F | | | | |
| | Q->pE/Q->pE, K Loss/K Loss, | 148209.8 | 148207.1 | −2.7 | −17.9 |
| | G1F/G2F | | | | |
| | Q->pE/Q->pE, K Loss/K Loss, | 147577.2 | 147583.7 | 6.5 | 43.9 |
| | G0F/G0 | | | | |
| | Q->pE/Q->pE, K Loss/K Loss, | 148371.9 | 148361.0 | −10.9 | −73.4 |
| | G2F/G2F | | | | |
| | Q->pE/Q->pE, K Loss/K Loss, | 147520.2 | 147513.6 | −6.6 | −44.4 |
| | G0F/G0F-GN | | | | |
| | Q->pE/Q->pE, K Loss/K Loss, | 147431.1 | 147432.6 | 1.5 | 10.4 |
| | G0/G0 | | | | |
| 2 | Q->pE/Q->pE, K Loss/K Loss, | 147885.5 | 147889.1 | 3.6 | 24.2 |
| | G0F/G1F | | | | |
| | Q->pE/Q->pE, K Loss/K Loss, | 148047.6 | 148049.9 | 2.2 | 15.0 |
| | G1F/G1F OR G0F/G2F | | | | |
| | Q->pE/Q->pE, K Loss/K Loss, | 147723.4 | 147729.1 | 5.8 | 39.2 |
| | G0F/G0F | | | | |
| | Q->pE/Q->pE, K Loss/K Loss, | 148209.8 | 148209.5 | −0.3 | −1.8 |
| | G1F/G2F | | | | |
| | Q->pE/Q->pE, K Loss/K Loss, | 147577.2 | 147583.5 | 6.2 | 42.3 |
| | G0F/G0 | | | | |
| | Q->pE/Q->pE, K Loss/K Loss, | 148371.9 | 148363.9 | −8.0 | −54.1 |
| | G2F/G2F | | | | |
| | Q->pE/Q->pE, K Loss/K Loss, | 147520.2 | 147513.6 | −6.6 | −44.4 |
| | G0F/G0F-GN | | | | |
| | Q->pE/Q->pE, K Loss/K Loss, | 147431.1 | 147437.7 | 6.6 | 45.0 |
| | G0/G0 | | | | |
| | LC/HC: Q->pE, K Loss, G1F | 74025.8 | 74025.4 | −0.4 | −5.8 |
| | LC/HC: Q->pE, K Loss, G0F | 73863.7 | 73864.0 | 0.3 | 4.1 |
| | LC/HC: Q->pE, K Loss, G2F | 74188.0 | 74187.2 | −0.8 | −10.6 |
| | LC/HC: Q->pE, K Loss, G0 | 73717.6 | 73716.3 | −1.3 | −17.3 |

Deglycosylated intact mass was obtained by diluting the samples with water, followed by enzymatic PNGase F digestion to remove the N-glycans, UHPLC separation, and Xevo G2-XS Q-TOF MS detection. The deconvoluted deglycosylated mass spectra data of the samples are shown in TABLE 3. The results show that the measured deglycosylation intact molecular weight for the DS samples are comparable, and are highly consistent with that of the theoretical deglycosylated molecule. However, peaks of half antibody (LC/HC) are again observed in ADS formulation 2 (TABLE 3-Sample ID 2, italicized), and there are no LC/HC peaks present in the ADS formulations 1 sample (TABLE 3-Sample ID 1) having increased (7.5-fold) trehalose.

TABLE 3

| | | Theor. | Measured | | Mass |
|---|---|---|---|---|---|
| | | Mass | Mass | Difference | Error |
| ID | Modification | (Da) | (Da) | (Da) | (ppm) |
| 1 | Deglycosylated, Q->pE/Q->pE, | 144834.7 | 144838.1 | 3.5 | 23.8 |
| | K Loss/K Loss | | | | |
| | Deglycosylated, Q->pE/Q->pE, | 144962.8 | 144971.7 | 8.9 | 61.3 |
| | K Loss | | | | |
| | Deglycosylated, Q->pE/Q->pE | 145091.0 | 145094.3 | 3.3 | 22.8 |
| 2 | Deglycosylated, Q->pE/Q->pE, | 144834.7 | 144838.5 | 3.9 | 26.9 |
| | K Loss/K Loss | | | | |
| | Deglycosylated, Q->pE/Q->pE, | 144962.8 | 144965.8 | 2.9 | 20.3 |
| | K Loss | | | | |
| | Deglycosylated, Q->pE/Q->pE | 145091.0 | 145093.8 | 2.8 | 19.0 |
| | LC/HC: Deglycosylated, Q->pE, | 72419.3 | 72420.7 | 1.3 | 18.3 |
| | K Loss | | | | |
| | LC/HC: Deglycosylated, Q->pE | 72547.5 | 72542.6 | −4.9 | −67.9 |

### Example 3—Molecular Species Distribution Analysis by SEC-MALS

Size exclusion chromatography-multi-angle light scattering (SEC-MALS) was used to determine the absolute molar mass and mean size of molecules in solution by determining the light scattering properties of species eluting from a sized-based chromatographic system. A MALS detector connected to an HPLC-SEC system was used to determine the molecular mass of the ADS samples (Formulations 1 and 2).

The static MALS instrument characterizes the absolute molecular weight of proteins based on the principle of static light scattering. The intensity of laser scattering is proportional to the molecular weight and protein concentration for proteins larger than 10 nm, and the molecular weight can be calculated according to Zimm's equation.

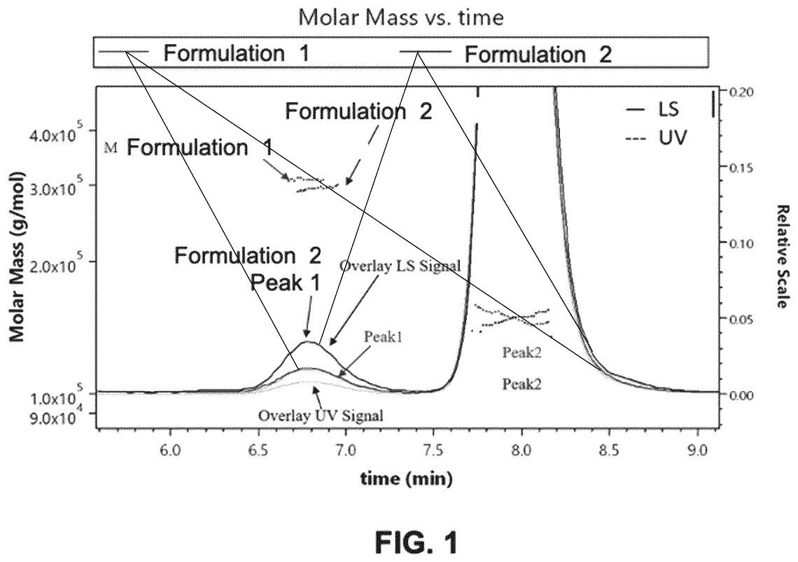

The SEC-MALS Chromatograms data of ADS samples are shown in TABLE 4 and FIG. 1. The molecular weight species (peak 1 and peak 2) was present in both samples. The molar mass of peak 1 and peak 2 was comparable for the two samples. The results from SEC-MALS show that the molecular weight of the main aggregate (peak 1) is in the range of 294 kDa to 308 kDa which may be dimer formed by 2 antibody protein molecules. ADS formulation 1 sample having increased (7.5-fold) trehalose exhibited less high molecular weight species/aggregates (i.e., less peak 1). Furthermore, light scattering and UV peak analysis showed that ADS formulation 1 exhibits a reduced aggregation peak (peak 1), wherein the aggregate peak 1 is reduced by a factor greater than 2 across both peak measurements. ADS formulation 1 reduces the aggregation by more than half.

TABLE 4

| | | Molar Mass | Light Scattering | UV Peak Statistics |
|---|---|---|---|---|
| ID | Peak | Mw (KDa) | Peak Area | Peak Area |
| 1 | 1 | 308.0 | 3.232 × 10\hat{ }−8 | 7.581 × 10\hat{ }−4 |
| | 2 | 148.7 | 3.568 × 10\hat{ }−6 | 1.278 × 10\hat{ }−1 |
| 2 | 1 | 294.5 | 6.624 × 10\hat{ }−8 | 17.46 × 10\hat{ }−4 |
| | 2 | 148.3 | 4.290 × 10\hat{ }−6 | 1.239 × 10\hat{ }−1 |

### Example 4—Thermal Stability by Differential Scanning Calorimetry

The thermal stability of ADS samples (formulations 1 and 2) was evaluated using differential scanning calorimetry (DSC), which determines a protein's thermal transition temperatures (Tm). Higher Tm values are indicative of increased thermal stability, and the temperature at which protein unfolding is initiated is the onset melting temperature (Tm onset). Tm and Tm onset are sensitive to changes in protein structure and stability and may be affected by differences in the formulation matrix.

DSC analysis of ADS formulation samples 1 and 2 was performed with a Malvern/MicroCal PEAQ DSC System. Samples were heated from 10° C. to 95° C. at a heating rate of 90° C./h. The DSC scanning results are shown in TABLE 5. ADS formulation sample 1 exhibited a higher Tm value, which can be attributed to the formulation.

TABLE 5

| | Test Result | | |
|---|---|---|---|
| ID | Tₘ Onset (° C.) | Tₘ₁ (° C.) | Tₘ₂ (° C.) |
| 1 | 64.6 | 69.1 | 85.9 |
| 2 | 60.0 | 66.4 | 84.7 |

### Example 5—Thermal Stress Testing

The ADS samples were tested in a thermal stress study. The ADS samples were thawed at room temperature and then transferred to a 1.5 mL microcentrifuge in the biosafety cabinet. The fill volume is 1 mL/vial. 4 vials for each ADS sample were prepared and one vial of each DS sample was used as TO control. The left vials were incubated in the stability chamber at 40° C. The duration, test points, and test items of the thermal stress study are listed in Table 6. At the end of each time point, the sample from each vial was aliquoted in 0.1 mL/vial in the biosafety cabinet and stored at −70±10° C.

Size exclusion—high-performance liquid chromatography (SE-HPLC) was used for monitoring the monomer, HMW, and LMW of ADS samples. 40 μg sample was injected into the Agilent 1260 system for the SE-HPLC. The HPLC system was equipped with a TSKGel G3000SW_{XL} column (5 μm, 7.8×300 mm) and a UV detector set at 280 nm. An isocratic gradient was applied at a 0.5 mL/min flow rate for 35 minutes.

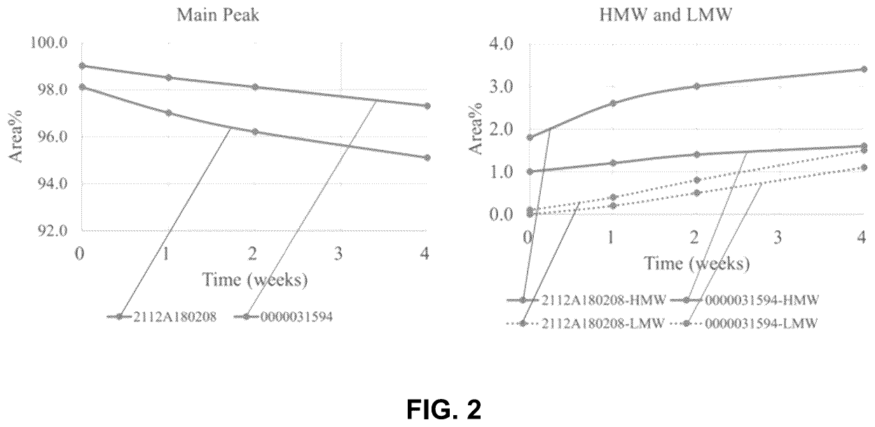

SE-HPLC results are summarized in TABLE 6. The results from SEC-HPLC show that ADS formulation 1 sample having increased (7.5-fold) trehalose and reduced sodium citrate buffer concentration (50 mM to 10 mM) exhibited less high molecular weight species/aggregates (i.e., HMW peak) as compared to the ADS formulation 2 sample. The mean difference was greater than 2-fold, wherein ADS formulation 1 reduces the mean aggregation by more than half when considering all timepoints. Furthermore, the % change in HMW Peak was 0.6% for ADS formulation 1, compared to 1.6% (2.6-fold greater) for ADS formulation 2. The same data is shown as a plot in FIG. 2. In FIG. 2, it is observed that the ADS formulation 2 sample exhibited an increased rate of aggregate formation over the course of the thermal stress study as compared to ADS formulation 1 sample, and exhibited a higher derivative dA/dt measuring the rate of aggregate formation over time.

In addition, the degradation rate of the drug product occurring during the thermal stress study was also measured using the SE-HPLC methodology. The results from SEC-HPLC show that ADS formulation 1 sample exhibited a decrease in purity of the drug product from 99% to 97.3% over the course of the study, while ADS formulation 2 sample exhibited a decrease in purity of the drug product from 98.1% to 95.1% over the course of the study. The mean difference between the rate of decrease of the drug product purity was approximately 1.6× greater in ADS formulation 2 sample as compared to ADS formulation 1 sample, with ADS formulation 1 sample exhibiting an approximate 40% decrease in the degradation rate of the drug product. The same data is shown as a plot in FIG. 2.

TABLE 6

| | Main Peak (%) | | | | HMW Peak (%) | | | |
|---|---|---|---|---|---|---|---|---|
| ID | T0 | 1 W | 2 W | 4 W | T0 | 1 W | 2 W | 4 W |
| 1 | 99.0 | 98.5 | 98.1 | 97.3 | 1.0 | 1.2 | 1.4 | 1.6 |
| 2 | 98.1 | 97.0 | 96.2 | 95.1 | 1.8 | 2.6 | 3.0 | 3.4 |

### Example 6—Free Sulfhydryl Analysis

Levels of free sulfhydryl in ADS samples were determined, as free sulfhydryl may indicate mismatched disulfide bonds and improper folding of the protein. A Thiol Assay Kit supplied was used to determine the free-SH concentration. This analysis involves the reaction of a fluorescence reagent with free sulfhydryl in the sample, resulting in fluorescence signals that correlated to the amount of free sulfhydryl.

ADS samples were diluted to 2-fold, 4-fold, and 8-fold in this experiment. The theoretical molecular weight of the ADS protein is 147723.4 Da, the free-SH concentration in the samples was calculated and the free-SH content of the ADS samples were 0.54% and 5.47%, respectively in TABLE 7. Levels of free sulfhydryl in ADS formulation sample 1 having increased (7.5-fold) trehalose were approximately 10-fold lower than the levels observed in ADS formulation sample 2, indicating more accurate protein folding in ADS formulation sample 1.

TABLE 7

| ID | Free-SH Content (μM/μM protein) |
|---|---|
| 1 | 0.54% |
| 2 | 5.47% |

While preferred embodiments of the present within a composition disclosure have been shown and described herein, it will be obvious to those skilled in the art that such embodiments are provided by way of example only. Numerous variations, changes, and substitutions will now occur to those skilled in the art without departing from the instant disclosure. It should be understood that various alternatives to the embodiments described herein may be employed in practicing the invention. It is intended that the following claims define the scope of the embodiments disclosed herein and that methods and structures within the scope of these claims and their equivalents be covered thereby.

SEQUENCES

| SEQ_ID | SEQUENCE | ANNOTATION |
|---|---|---|
| 1 | GYAFTAYN | H_CDR1 |
| 2 | FDPYDGGS | H_CDR2 |
| 3 | GWYYFDY | H_CDR3 |
| 4 | KSISKY | L_CDR1 |
| 5 | SGS | L_CDR2 |
| 6 | QQHDESPY | L_CDR3 |
| 7 | QVQLQESGPGLVKPSQTLSLTCTVSGYAFTAYNIHWV | H_VAR |
| | RQAPGQGLEWMGSFDPYDGGSSYNQKFKDRLTISKDT | |
| | SKNQVVLTMTNMDPVDTATYYCARGWYYFDYWGHG | |
| | TLVTVSS | |
| 8 | DIVMTQTPLSLPVTPGEPASISCRASKSISKYLAWYQQK | L_VAR |
| | PGQAPRLLIYSGSTLQSGIPPRFSGSGYGTDFTLTINNIES | |
| | EDAAYYFCQQHDESPYTFGEGTKVEIKRTV | |
| 9 | QVQLQESGPGLVKPSQTLSLTCTVSGYAFTAYNIHWV | H_FULL |
| | RQAPGQGLEWMGSFDPYDGGSSYNQKFKDRLTISKDT | |
| | SKNQVVLTMTNMDPVDTATYYCARGWYYFDYWGHG | |
| | TLVTVSSASTKGPSVFPLAPSSKSTSGGTAALGCLVKD | |
| | YFPEPVTVSWNSGALTSGVHTFPAVLQSSGLYSLSSVV | |
| | TVPSSSLGTQTYICNVNHKPSNTKVDKKVEPKSCDKTH | |
| | TCPPCPAPELLGGPSVFLFPPKPKDTLMISRTPEVTCVV | |
| | VDVSHEDPEVKFNWYVDGVEVHNAKTKPREEQYNST | |
| | YRVVSVLTVLHQDWLNGKEYKCKVSNKALPAPIEKTI | |
| | SKAKGQPREPQVYTLPPSRDELTKNQVSLTCLVKGFYP | |
| | SDIAVEWESNGQPENNYKTTPPVLDSDGSFFLYSKLTV | |
| | DKSRWQQGNVFSCSVMHEALHNHYTQKSLSLSPGK | |
| 10 | DIVMTQTPLSLPVTPGEPASISCRASKSISKYLAWYQQK | L_FULL |
| | PGQAPRLLIYSGSTLQSGIPPRFSGSGYGTDFTLTINNIES | |
| | EDAAYYFCQQHDESPYTFGEGTKVEIKRTVAAPSVFIF | |
| | PPSDEQLKSGTASVVCLLNNFYPREAKVQWKVDNALQ | |
| | SGNSQESVTEQDSKDSTYSLSSTLTLSKADYEKHKVYA | |
| | CEVTHQGLSSPVTKSFNRGEC | |
| 11 | ASTKGPSVFPLAPSSKSTSGGTAALGCLVKDYFPEPVT | H_CONSTANT |
| | VSWNSGALTSGVHTFPAVLQSSGLYSLSSVVTVPSSSL | |
| | GTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYNSTYRVVSV | |
| | LTVLHQDWLNGKEYKCKVSNKALPAPIEKTISKAKGQ | |
| | PREPQVYTLPPSRDELTKNQVSLTCLVKGFYPSDIAVE | |
| | WESNGQPENNYKTTPPVLDSDGSFFLYSKLTVDKSRW | |
| | QQGNVFSCSVMHEALHNHYTQKSLSLSPGK | |

1. A composition comprising:
an ROR1 antibody or antigen binding fragment thereof at a concentration of between about 20 and about 80 milligrams per milliliter;
a buffer;
a chelating agent;
a surfactant; and
5.0% (w/v) or greater of trehalose dihydrate;
wherein the pH of the composition is 6.0 or lower.

2. The composition of claim 1, wherein the composition reduces the formation of aggregates.

3. The composition of claim 2, wherein the aggregates comprise precipitates, wherein the wherein the composition reduces the formation precipitates.

4. The composition of claim 1, wherein the composition comprises less than 2% of antibody aggregates.

5. The composition of claim 1, wherein the composition comprises less than 2% of antibody aggregates after incubation at 40 C for 4 weeks.

6. The composition of claim 1, wherein the composition maintains the purity of the antibody or antigen binding fragment thereof.

7. The composition of claim 1, wherein the composition maintains at least 97% purity of the antibody or antigen binding fragment thereof.

8. The composition of claim 1, wherein the composition maintains at least 97% purity of the antibody or antigen binding fragment thereof after incubation at 40 C for 4 weeks.

9. The composition of claim 1, wherein the antibody is zilovertamab.

10. The composition of any one of claims 1 to 9, wherein the buffer comprises acetate, citrate, histidine, arginine, succinate, or phosphate.

11. The composition of claim 10, wherein the buffer comprises sodium citrate.

12. The composition of any one of claims 1 to 11, wherein the buffer is present within the composition at a concentration from about 5 mM to about 100 mM.

13. The composition of any one of claims 1 to 11, wherein the buffer is present within the composition at a concentration from about 5 mM to about 50 mM.

14. The composition of any one of claims 1 to 11, wherein the buffer is present within the composition at a concentration from about 5 mM to about 20 mM.

15. The composition of any one of claims 1 to 11, wherein the buffer is present within the composition at a concentration of about 10 mM.

16. The composition of any one of claims 1 to 11, wherein the buffer is present within the composition at a concentration of up to 10 mM.

17. The composition of claim 16, wherein the buffer is sodium citrate.

18. The composition of any one of claims 1 to 15, wherein the chelating agent comprises ethylenediaminetetraacetic acid (EDTA) or diethylenetriamine pentaacetate (DTPA).

19. The composition of claim 18, wherein the chelating agent comprises EDTA.

20. The composition of any one of claims 1 to 19, wherein the chelating agent is present within the composition at a concentration from about 0.01 mM to about 0.2 mM.

21. The composition of any one of claims 1 to 19, wherein the chelating agent is present within the composition at a concentration from about 0.01 mM to about 0.1 mM.

22. The composition of any one of claims 1 to 19, wherein the chelating agent is present within the composition at a concentration from about 0.02 mM to about 0.2 mM.

23. The composition of any one of claims 1 to 19, wherein the chelating agent is present within the composition at a concentration of about 0.05 mM.

24. The composition of any one of claims 1 to 23, wherein the surfactant comprises polysorbate 80, polysorbate 20, or a polyethylene-polypropylene copolymer.

25. The composition of any one of claims 1 to 23, wherein the surfactant comprises polysorbate 80.

26. The composition of any one of claims 1 to 25, wherein the surfactant is present within the composition at a concentration from about 0.005% (w/v) to about 0.1% (w/v).

27. The composition of any one of claims 1 to 25, wherein the surfactant is present within the composition at a concentration from about 0.01% (w/v) to about 0.05% (w/v).

28. The composition of any one of claims 1 to 25, wherein the surfactant is present within the composition at a concentration from about 0.01% (w/v) to about 0.03% (w/v).

29. The composition of any one of claims 1 to 25, wherein the surfactant is present within the composition at a concentration of about 0.02% (w/v).

30. The composition of any one of claims 1 to 29, wherein the trehalose dihydrate is present within the composition at a concentration from about 5.0% (w/v) to about 10.0% (w/v).

31. The composition of any one of claims 1 to 29, wherein the trehalose dihydrate is present within the composition at a concentration from about 6.0% (w/v) to about 9.0% (w/v).

32. The composition of any one of claims 1 to 29, wherein the trehalose dihydrate is present within the composition at a concentration from about 7.0% (w/v) to about 8.0% (w/v).

33. The composition of any one of claims 1 to 29, wherein the trehalose dihydrate is present within the composition at a concentration of about 7.5% (w/v).

34. The composition of any one of claims 1 to 33, wherein the pH is from about 4.0 to about 6.0.

35. The composition of any one of claims 1 to 33, wherein the pH is from about 4.5 to about 6.0.

36. The composition of any one of claims 1 to 33, wherein the pH is from about 4.0 to about 5.5.

37. The composition of any one of claims 1 to 33, wherein the pH is from about 4.5 to about 5.5.

38. The composition of any one of claims 1 to 33, wherein the pH is from about 5.0 to about 5.5.

39. The composition of any one of claims 1 to 33, wherein the pH is about 5.2.

40. The composition of any one of claims 1 to 39, wherein the composition is a liquid.

41. A method of treating a cancer comprising administering the composition of any one of claims 1 to 40 to an individual in need thereof.

42. The method of claim 41, wherein the cancer is a leukemia or lymphoma.

43. The method of claim 42, wherein the leukemia or lymphoma is B cell leukemia or lymphoma.

44. The method of claim 42, wherein the leukemia or lymphoma is chronic lymphocytic leukemia (CLL).

45. The method of claim 42, wherein the leukemia or lymphoma is mantle cell lymphoma (MCL).

46. The method of claim 41, wherein the cancer is a solid tissue cancer.

47. The method of claim 46, wherein the solid tissue cancer is breast cancer, ovarian cancer, prostate cancer, lung cancer, pancreatic cancer, head and neck cancer, kidney cancer, colon cancer, or stomach cancer.

48. A composition comprising:
an ROR1 antibody or antigen binding fragment thereof at a concentration of about 40 milligrams per milliliter;
about 10 mM sodium citrate;
about 0.05 mM EDTA;
about 0.02% (w/v) polysorbate 80; and
about 7.5% (w/v) trehalose dihydrate;
wherein the pH of the composition is about 5.2.

49. The composition of claim 48, wherein the composition reduces the formation of aggregates.

50. The composition of claim 49, wherein the aggregates comprise precipitates, wherein the wherein the composition reduces the formation precipitates.

51. The composition of claim 48, wherein the composition comprises less than 2% of antibody aggregates.

52. The composition of claim 48, wherein the composition comprises less than 2% of antibody aggregates after incubation at 40 C for 4 weeks.

53. The composition of claim 48, wherein the composition maintains the purity of the antibody or antigen binding fragment thereof.

54. The composition of claim 48, wherein the composition maintains at least 97% purity of the antibody or antigen binding fragment thereof.

55. The composition of claim 48, wherein the composition maintains at least 97% purity of the antibody or antigen binding fragment thereof after incubation at 40 C for 4 weeks.

56. The composition of claim 48, wherein the antibody is zilovertamab.

57. A composition comprising:
an anti-ROR1 antibody or ROR1-binding antibody fragment thereof at a concentration of between about 20 and about 80 milligrams per milliliter (mg/mL); and
about 5% (w/v) or greater trehalose;
wherein the anti-ROR1 antibody or ROR1-binding antibody fragment thereof comprises:
(a) a heavy chain complementarity determining region 1 (H-CDR1) comprising an amino acid sequence set forth in SEQ ID NO: 1;
(b) a heavy chain complementarity determining region 2 (H-CDR2) comprising an amino acid sequence set forth in SEQ ID NO: 2;
(c) a heavy chain complementarity determining region 3 (H-CDR3) comprising an amino acid sequence set forth in SEQ ID NO: 3;
(d) a light chain complementarity determining region 1 (L-CDR1) comprising an amino acid sequence set forth in SEQ ID NO: 4;
(e) a light chain complementarity determining region 2 (L-CDR2) comprising an amino acid sequence set forth in SEQ ID NO: 5; and/or
(f) a light chain complementarity determining region 3 (L-CDR3) comprising an amino acid sequence set forth in SEQ ID NO: 6;
wherein the anti-ROR1 antibody or ROR1-binding antibody fragment thereof binds ROR1.

58. The composition of claim 57, wherein the composition reduces the formation of aggregates.

59. The composition of claim 58, wherein the aggregates comprise precipitates, wherein the wherein the composition reduces the formation precipitates.

60. The composition of claim 57, wherein the composition comprises less than 2% of antibody aggregates.

61. The composition of claim 57, wherein the composition comprises less than 2% of antibody aggregates after incubation at 40 C for 4 weeks.

62. The composition of claim 57, wherein the composition maintains the purity of the antibody or antigen binding fragment thereof.

63. The composition of claim 57, wherein the composition maintains at least 97% purity of the antibody or antigen binding fragment thereof.

64. The composition of claim 57, wherein the composition maintains at least 97% purity of the antibody or antigen binding fragment thereof after incubation at 40 C for 4 weeks.

65. The composition of claim 57, wherein the anti-ROR1 antibody or ROR1-binding antibody fragment thereof comprises:
a heavy chain variable domain comprising 90% or greater sequence identity to SEQ ID NO: 7; and
a light chain variable domain comprising 90% or greater sequence identity to SEQ ID NO: 8.

66. The composition of claim 57, wherein the anti-ROR1 antibody or ROR1-binding antibody fragment thereof comprises:
a heavy chain variable domain comprising SEQ ID NO: 7; and
a light chain variable domain comprising SEQ ID NO: 8.

67. The composition of claim 57, wherein the anti-ROR1 antibody or ROR1-binding antibody fragment thereof comprises:
a heavy chain constant domain comprising 90% or greater sequence identity to SEQ ID NO: 11

68. The composition of claim 57, wherein the anti-ROR1 antibody or ROR1-binding antibody fragment thereof comprises:
a heavy chain domain comprising 90% or greater sequence identity to SEQ ID NO: 9; and
a light chain domain comprising 90% or greater sequence identity to SEQ ID NO: 10.

69. The composition of claim 57, wherein the anti-ROR1 antibody or ROR1-binding antibody fragment thereof comprises:
a heavy chain domain comprising SEQ ID NO: 9; and
a light chain domain comprising SEQ ID NO: 10.

70. The composition of any one of claims 57 to 69, wherein the anti-ROR1 antibody is zilovertamab.

71. The composition of any one of claims 57 to 70, wherein the anti-ROR1 antibody or ROR1-binding antibody fragment thereof is present at a concentration of between about 30 mg/mL to about 50 mg/mL.

72. The composition of claim 71, wherein the anti-ROR1 antibody or ROR1-binding antibody fragment thereof is present at a concentration of about 40 mg/mL.

73. The composition of any one of claims 57 to 69, wherein the composition comprises about 7% (w/v) or greater trehalose.

74. The composition of claim 52, wherein the composition comprises about 7.5% (w/v) trehalose.

75. The composition of any one of claims 57 to 69, wherein the composition further comprises a citrate buffer.

76. The composition of claim 69, wherein the composition comprises less than about 20 mM citrate buffer.

77. The composition of claim 69, wherein the composition comprises less than about 15 mM citrate buffer.

78. The composition of claim 69, wherein the composition comprises between about 5 mM to about 20 mM citrate buffer.

79. The composition of claim 69, wherein the composition comprises about 10 mM citrate buffer.

80. The composition of claim 69, wherein the composition comprises up to 10 mM citrate buffer.

81. The composition of claim 80, wherein the citrate buffer is sodium citrate.

82. The composition of any one of claims 57 to 81, wherein the composition is buffered to comprise a pH between about 4.5 to about 5.5.

83. The composition of any one of claims 57 to 82, wherein the composition further comprises a non-ionic surfactant.

84. The composition of claim 83, wherein the non-ionic surfactant is polysorbate 80.

85. The composition of claim 84, wherein the composition comprises about 0.010% (w/v) to about 0.050% (w/v) polysorbate 80.

86. The composition of any one of claims 57 to 85, wherein the composition further comprises a chelator.

87. The composition of claim 86, wherein the chelator comprises EDTA.

88. The composition claim 86, wherein the composition comprises:
an anti-ROR1 antibody or ROR1-binding antibody fragment thereof at a concentration of about 40 mg/mL;
about 7.5% (w/v) trehalose dihydrate;
about 10 mM citrate buffer;
about 0.02% (w/v) polysorbate 80; and
about 0.05 mM EDTA.

89. The composition of claim 88, wherein the citrate buffer is sodium citrate.


SEQUENCE LISTING (11 sequences)

SEQ ID NO: 1 - protein, 8 aa, synthetic construct
GYAFTAYN

SEQ ID NO: 2 - protein, 8 aa, synthetic construct
FDPYDGGS

SEQ ID NO: 3 - protein, 7 aa, synthetic construct
GWYYFDY

SEQ ID NO: 4 - protein, 6 aa, synthetic construct
KSISKY

SEQ ID NO: 5 - skipped

SEQ ID NO: 6 - protein, 8 aa, synthetic construct
QQHDESPY

SEQ ID NO: 7 - protein, 116 aa, synthetic construct
QVQLQESGPGLVKPSQTLSLTCTVSGYAFTAYNIHWVRQAPGQGLEWMGSFDPYDGGSSYNQKFKDRLTISKDTSKNQVVLTMTNMDPVDTATYYCARGWYYFDYWGHGTLVTVSS

SEQ ID NO: 8 - protein, 110 aa, synthetic construct
DIVMTQTPLSLPVTPGEPASISCRASKSISKYLAWYQQKPGQAPRLLIYSGSTLQSGIPPRFSGSGYGTDFTLTINNIESEDAAYYFCQQHDESPYTFGEGTKVEIKRTV

SEQ ID NO: 9 - protein, 446 aa, synthetic construct
QVQLQESGPGLVKPSQTLSLTCTVSGYAFTAYNIHWVRQAPGQGLEWMGSFDPYDGGSSYNQKFKDRLTISKDTSKNQVVLTMTNMDPVDTATYYCARGWYYFDYWGHGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct
DIVMTQTPLSLPVTPGEPASISCRASKSISKYLAWYQQKPGQAPRLLIYSGSTLQSGIPPRFSGSGYGTDFTLTINNIESEDAAYYFCQQHDESPYTFGEGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 330 aa, synthetic construct [sequence omitted]